(19) United States
(12) Patent Application Publication
(10) Pub. No.: US 2013/0217710 A1
(43) Pub. Date: Aug. 22, 2013

(54) METHODS FOR TREATING CANCER

(71) Applicants: Manuel Carlos Alves-Aivado, Collegeville, PA (US); Gautam Borthakur, Houston, TX (US); Tapan M. Kadia, Houston, TX (US); Hagop M. Kantarjian, Houston, TX (US); Sylvie Laquerre, King of Prussia, PA (US)

(72) Inventors: Manuel Carlos Alves-Aivado, Collegeville, PA (US); Gautam Borthakur, Houston, TX (US); Tapan M. Kadia, Houston, TX (US); Hagop M. Kantarjian, Houston, TX (US); Sylvie Laquerre, King of Prussia, PA (US)

(73) Assignee: GLAXOSMITHKLINE INTELLECTUAL PROPERTY (NO.2) LIMITED, Brentford (GB)

(21) Appl. No.: 13/883,374

(22) PCT Filed: Nov. 4, 2011
(86) PCT No.: PCT/US11/59285

§ 371 Date: May 3, 2013

Related U.S. Application Data
(60) Provisional application No. 61/410,560, filed on Nov. 5, 2010.
(60) Provisional application No. 61/422,715, filed on Dec. 14, 2010.

(51) Int. Cl. A61K 31/519; A61K 31/436; A61K 45/06; A61K 31/506

(52) U.S. Cl. CPC A61K 31/519; A61K 31/506; A61K 31/436; A61K 45/06
USPC 514/264.1

(57) ABSTRACT

Methods are provided for treating a human having cancer comprising detecting at least one mutation in a Ras protein or a gene encoding at least one Ras protein from at least one tumor cell from said human and treating said human having at least one mutation in at least one Ras protein or a gene encoding at least one Ras protein with a pharmaceutical composition comprising at least one MEK inhibitor comprising a compound of Structure (I):

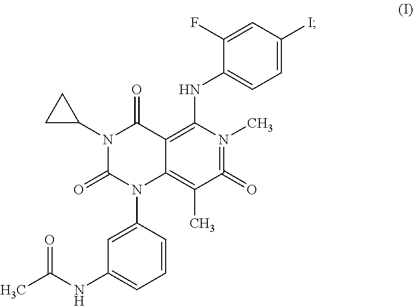

or a pharmaceutically acceptable salt or solvate thereof.

### FIELD OF THE INVENTION

The present invention relates to a method of treating cancer in a mammal. In particular, the method relates to methods comprising treating a human having at least one mutation in a Ras protein and/or at least one gene encoding at least one Ras protein from at least one tumor cell comprising administering the MEK inhibitor: N-{3-[3-cyclopropyl-5-(2-fluoro-4-iodo-phenylamino)6,8-dimethyl;-2,4,7-trioxo-3,4,6,7-tetrahydro-2H-pyrido[4,3-d]pyrimidin-1-yl]phenyl}acetamide, or a pharmaceutically acceptable salt or solvate thereof to said human.

### BACKGROUND OF THE INVENTION

Effective treatment of hyperproliferative disorders, including cancer, is a continuing goal and unmet medical need in the oncology field. Generally, cancer results from the deregulation of the normal processes that control cell growth, cell division, differentiation and apoptotic cell death, among others. One such process involves kinase regulation of apoptosis and cellular signaling from growth factor receptors at the cell surface to the nucleus (Crews and Erikson, Cell, 74:215-17, 1993).

A large family of enzymes is the protein kinase enzyme family. There are about 500 different known protein kinases. Protein kinases serve to catalyze the phosphorylation of an amino acid side chain in various proteins by the transfer of the γ-phosphate of the ATP-Mg²⁺ complex to said amino acid side chain. These enzymes appear to control the majority of the signaling processes inside cells, thereby governing cell function, growth, differentiation and apoptosis through reversible phosphorylation of the hydroxyl groups of serine, threonine and tyrosine residues in proteins. Studies have shown that protein kinases regulate many cell functions, including signal transduction, transcriptional regulation, cell motility, and cell division. Several oncogenes have also been shown to encode protein kinases, suggesting that kinases play a role in oncogenesis.

For example, activation of Raf-MEK-ERK signal transduction pathway in cancer, particularly colorectal cancer, pancreatic cancer, lung cancer, breast cancer and the like, has been observed.

The ras family of oncogenes (K-ras, H-ras, and N-ras) encode for membrane proteins possessing GTPase activity. These proteins are involved in cellular signal transduction. Specific point mutations, usually within the ras codons 12, 13, or 61, can result in the activation of these protooncogenes and result in subsequent neoplasia (Bos, J. L., 1989, Can. Res. 49:4682-4689). The frequency with which ras mutations occur varies among different tumor types, although not all have been tested. Studies indicate that approximately 40-50% of colon cancers exhibit a mutation in the c-K-ras gene, with 86% of these mutations occurring at codons 12 and 13 (Bos, J. L. et al., 1987, Nature 327: (6120)293-7, Vogelstein B. et al., 1988, N. Engl. J. Med. 319:525-532). Ras mutations result in increased cell proliferation due to decreased intrinsic GTP-ase activity of the Ras protein.

It would be useful to provide novel methods of treatment for an individual with cancer having at least one Ras protein mutation.

### BRIEF DESCRIPTION OF THE DRAWINGS

FIG. 1 Sensitivity (gIC50) of Haematopoietic Cancer Cell Lines to MEK Inhibitor, Compound A.

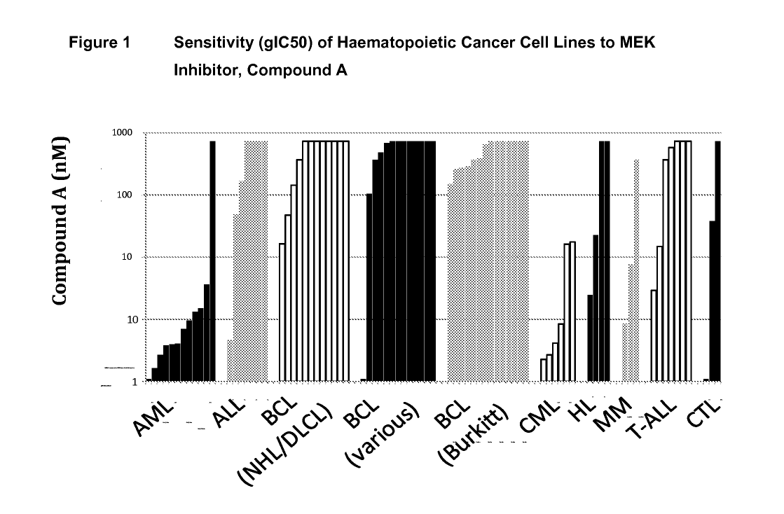

FIG. 2 Combination Effect of MEK Inhibitor (Compound A) with Different Drugs on Haematopoietic Cancer Cell Lines of AML Origin.

FIG. 3: Peripheral Blasts, Platelets, and White Blood Cell Count after Treatment with Compound A for 74 yo F with T-MDS>AML, Prior treatment: DAC>CR 15 months.

### SUMMARY OF THE INVENTION

In one embodiment of the present invention methods are provided for treating a mammal having cancer comprising detecting at least one mutation in a Ras protein or a gene encoding at least one Ras protein from at least one tumor cell from said mammal and treating said mammal having at least one mutation in at least one Ras protein or a gene encoding at least one Ras protein with a pharmaceutical composition comprising at least one MEK inhibitor comprising a compound of Structure (I):

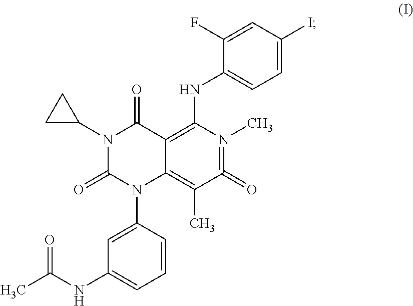

or a pharmaceutically acceptable salt or solvate thereof. In one embodiment, the mammal is human.

### DETAILED DESCRIPTION OF THE INVENTION

In one embodiment of the present invention methods are provided for treating a mammal having cancer comprising detecting at least one mutation in a Ras protein or a gene encoding at least one Ras protein from at least one tumor cell from said mammal and treating said mammal having at least one mutation in at least one Ras protein or a gene encoding at least one Ras protein with a pharmaceutical composition comprising at least one MEK inhibitor comprising a compound of Structure (I):

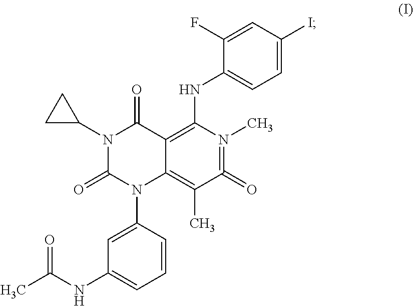

or a pharmaceutically acceptable salt or solvate or thereof. In one aspect the mammal is human.

The cancer may be any cancer in which an abnormal number of blast cells are present or that is diagnosed as a haematological cancer or dysplasia, such as leukemia, myeloid malignancy or myeloid dysplasia, including but not limited to, undifferentiated acute myelogenous leukemia, myeloblastic leukemia, myeloblastic leukemia, promyelocytic leukemia, myelomonocytic leukemia, monocytic leukemia, erythroleukemia and megakaryoblastic leukemia. In one aspect, the cancer is a myeloid malignancy cancer. In another aspect, the cancer is leukemia. The leukemia may be acute lymphocytic leukemia, acute non-lymphocytic leukemia, acute myeloid leukemia (AML), chronic lymphocytic leukemia, chronic myelogenous (or myeloid) leukemia (CML), and chronic myelomonocytic leukemia (CMML). In one embodiment, the human has agnogenic myeloid metaplasia and/or poor-risk myelodysplasia (MDS). In some aspects the cancer is relapsed or refractory. Patients may have received one or more treatments for leukemia prior to receiving Structure I.

In another embodiment, Structure (I), also referred to as N-{3-[3-cyclopropyl-5-(2-fluoro-4-iodo-phenylamino)-6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate thereof (hereinafter Compound A, or a pharmaceutically acceptable salt or solvate thereof) is in a sodium salt form. In another aspect, Compound A is in the form of a dimethyl sulfoxide solvate.

N-{3-[3-cyclopropyl-5-(2-fluoro-4-iodo-phenylamino)-6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate is a highly selective allosteric inhibitor of mitogen activated extracellular signal-regulated kinase 1 (MEK 1) and MEK 2. MEK proteins are a node in a certain extracellular signal-related kinase ERK pathway which is commonly hyper-activated in tumor cells. Oncogenic mutations in both B-raf and Ras signal through MEK1 and MEK2. In vitro, 80% of cell lines carrying activating mutations of B-Raf and 72% of Ras mutant cell lines were sensitive to N-{3-[3-cyclopropyl-5-(2-fluoro-4-iodo-phenylamino)-6,8-dimethyl-2,4,7-trioxo-3,4,6,7-tetrahydro-2H-pyrido[4,3-d]pyrimidin-1-yl]phenyl}acetamide, or a pharmaceutically acceptable salt or solvate in cell proliferation assays, and a majority (83% of hematopoietic cancers from acute or chronic myeloid leukemia (AML or CML, respectively) origins were also very sensitive.

In another embodiment, the present invention provides methods for treating cancer comprising administering at least one Braf inhibitor with Compound A, or a pharmaceutically acceptable salt or solvate thereof. In one aspect, the Braf inhibitor is Structure (II):

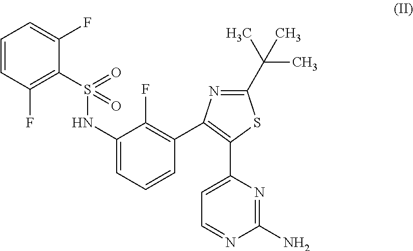 [SMILES: CC(C)(C)c1nc(-c2cccc(NS(=O)(=O)c3c(F)cccc3F)c2F)c(-c2ccnc(N)n2)s1]

or a pharmaceutically acceptable salt thereof, also referred to as N N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate or a pharmaceutically acceptable salt thereof, (hereinafter Compound B or a pharmaceutically acceptable salt thereof).

In another aspect of the present invention, methods are provided for treating a human with leukemia and having at least one mutation in a Ras protein or a gene encoding at least one Ras protein from at least one tumor cell with a pharmaceutical composition comprising Structure I or a pharmaceutically acceptable salt or solvate thereof and at least one of the following: at least one mTOR inhibitor, rapamycin, ara-C, bexarotene, and sorafenib. In some instances the mTOR inhibitor can be selected from rapamycin, rapalogs, everolimus, deforolimus, and temsirolimus.

In one aspect, at least one Ras mutation in said tumor cell occurs in exon 2 and/or exon 3. In another embodiment, the mutation in at least one Ras protein or gene encoding at least one Ras protein is in K-ras, H-ras and/or N-ras. The gene encoding at least one Ras protein may have a mutation in at least one of Ras codons 12, 13, 14, 59, 61, 74, 76 and 146. In some aspects the Ras protein has a mutation selected from G12S, G12V, G12D, G12A, G12C, G12R, G13A, G13D, G13R, V14I, G60E, Q61H, Q61K, Q61R, T74P, E76G, E76K, E76Q and A146T. In some aspects, the mutation in Kras is G12A and/or the mutation in N-ras is G12S.

In another aspect the mammal also has a Braf mutation. In some instances, the Braf mutation is selected from R462I, I463S, G464V, G464E, G466A, G466E, G466V, G469A, G469E, D594V, F595L, G596R, L597V, L597R, T599I, V600E, V600D, V600K, V600R, T119S, and K601E. In some instances, the Braf mutation is detected in the same tumor cell and/or the same type of tumor cell as a Ras mutation.

In another embodiment, the human shows a complete remission of a myeloid malignancy with administration of a pharmaceutical composition comprising Structure I or a pharmaceutically acceptable salt or solvate thereof. Complete remission can mean that a human is free of all, or substantially all, symptoms of leukemia and/or has an absolute neutrophil count ≧1×10⁹/L and/or has a platelet count ≧100×10⁹/L and/or normal marrow differential with ≦5% blast cells. In some aspects, the human has no blast cells in bone marrow after receiving a pharmaceutical composition comprising Structure I or a pharmaceutically acceptable salt or solvate thereof. In some aspects the human receives at least one week of treatment of a pharmaceutical composition comprising Structure I or a pharmaceutically acceptable salt or solvate thereof. As is contemplated by this invention, a human who receives a pharmaceutical composition comprising Structure I or a pharmaceutically acceptable salt or solvate thereof and presents 5% blast cells in bone marrow and/or complete remission can maintain blast cell counts less than 5% for a certain interim while remaining on treatment. For example, blast cells may be reduced or eliminated with treatment and remain reduced or eliminated for a week, a month, several months or longer and/or for the duration of treatment and/or after treatment has finished. In some aspects the human shows no blast cells in bone marrow or an undetectable amount after receiving about one week of treatment with a pharmaceutical composition comprising Structure I or a pharmaceutically acceptable salt or solvate thereof. In some instances, the patient remains on therapy for at least 8 weeks.

In another embodiment, the amount of Structure I or a pharmaceutically acceptable salt or solvate thereof administered to said human is an amount selected from 0.125 mg to 10 mg. In some aspects the amount of Structure I or a pharmaceutically acceptable salt or solvate thereof administered to said human is administered daily from about 1 mg/day to about 2 mg/day. The amount of BRAF inhibitor is an amount selected from 75 mg to 1,000 mg. In some aspects the pharmaceutical composition comprises N N-{3-[5-(2-Amino-4-pyrimidinyl)-2-(1,1-dimethylethyl)-1,3-thiazol-4-yl]-2-fluorophenyl}-2,6-difluorobenzenesulfonamide methanesulfonate or a pharmaceutically acceptable salt thereof, (hereinafter Compound B or a pharmaceutically acceptable salt thereof) in an amount from about 75 mg to about 1,000 mg. In some aspects, the pharmaceutical composition comprising Structure I or a pharmaceutically acceptable salt or solvate thereof and the pharmaceutical composition comprising at least one Braf inhibitor are administered separately. In another aspect, the pharmaceutical composition comprising Structure I or a pharmaceutically acceptable salt or solvate thereof is administered at the same time as the pharmaceutical composition comprising Structure II or a pharmaceutically acceptable salt or solvate thereof.

Compound A is disclosed and claimed, along with pharmaceutically acceptable salts and solvates thereof, as being useful as an inhibitor of MEK activity, particularly in treatment of cancer, in International Application No. PCT/JP2005/011082, having an International filing date of Jun. 10, 2005; International Publication Number WO 2005/121142 and an International Publication date of Dec. 22, 2005, the entire disclosure of which is hereby incorporated by reference, Compound A is the compound of Example 4-1. Compound A can be prepared as described in International Application No. PCT/JP2005/011082. Compound A can be prepared as described in United States Patent Publication No. US 2006/0014768, Published Jan. 19, 2006, the entire disclosure of which is hereby incorporated by reference.

Suitably, Compound A is in the form of a dimethyl sulfoxide solvate. Suitably, Compound A is in the form of a sodium salt. Suitably, Compound A is in the form of a solvate selected from: hydrate, acetic acid, ethanol, nitromethane, chlorobenzene, 1-pentanci, isopropyl alcohol, ethylene glycol and 3-methyl-1-butanol. These solvates and salt forms can be prepared by one of skill in the art from, for example, the description in International Application No. PCT/JP2005/011082 or United States Patent Publication No. US 2006/0014768.

Compound B is disclosed and claimed, along with pharmaceutically acceptable salts thereof, as being useful as an inhibitor of BRaf activity, particularly in the treatment of cancer, in PCT patent application PCT/US09/42682. Compound B is embodied therein by Examples 58a through 58e of the application. This PCT application was published on 12 Nov. 2009 as publication WO2009/137391, and is hereby incorporated by reference.

The compounds of the invention may contain one or more chiral atoms, or may otherwise be capable of existing as two enantiomers. Accordingly, the compounds of this invention include mixtures of enantiomers as well as purified enantiomers or enantiomerically enriched mixtures. Also, it is understood that all tautomers and mixtures of tautomers are included within the scope of Compound A, and pharmaceutically acceptable salts s thereof, and Compound B, and pharmaceutically acceptable salts thereof.

The compounds of the invention may form a solvate which is understood to be a complex of variable stoichiometry formed by a solute (in this invention, Compound A or a salt thereof and/or Compound B or a salt thereof) and a solvent. Such solvents for the purpose of the invention may not interfere with the biological activity of the solute. Examples of suitable solvents include, but are not limited to, water, methanol, dimethyl sulfoxide, ethanol and acetic acid. Suitably the solvent used is a pharmaceutically acceptable solvent. Examples of suitable pharmaceutically acceptable solvents include, without limitation, water, dimethyl sulfoxide, ethanol and acetic acid. Suitably the solvent used is water.

The pharmaceutically acceptable salts of the compounds of the invention are readily prepared by those of skill in the art.

Also, contemplated herein is a method of treating cancer using a combination of the invention where Compound A, or a pharmaceutically acceptable salt or solvate thereof, and/or Compound B or a pharmaceutically acceptable salt thereof are administered as pro-drugs. Pharmaceutically acceptable pro-drugs of the compounds of the invention are readily prepared by those of skill in the art.

By the term “treating” and grammatical variations thereof as used herein, is meant therapeutic therapy. In reference to a particular condition, treating means: (1) to ameliorate or prevent the condition of one or more of the biological manifestations of the condition, (2) to interfere with (a) one or more points in the biological cascade that leads to or is responsible for the condition or (b) one or more of the biological manifestations of the condition, (3) to alleviate one or more of the symptoms, effects or side effects associated with the condition or treatment thereof, or (4) to slow the progression of the condition or one or more of the biological manifestations of the condition. Prophylactic therapy is also contemplated thereby. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof. Prophylactic therapy is appropriate, for example, when a subject is considered at high risk for developing cancer, such as when a subject has a strong family history of cancer or when a subject has been exposed to a carcinogen.

In patients with Acute Leukemias, including poor-risk myelodysplasia, poor-risk myelodysplasia-BP, chronic myelomonocytic leukemia (Cheson, et al. Blood. 2000; 96: 3671-3674; Cheson, et al. J Clin Oncol. 2003; 21:4642-4649; Cheson, et al. Blood 2006; 108: 419-425) the following response definitions can be used.

As used herein the term “complete remission” is used to describe outcomes for subjects with poor-risk myelodysplasia, chronic myelogenous leukemia, chronic lymphocytic leukemia, and acute leukemia and means a patient is free of all symptoms related to leukemia and has an absolute neutrophil count ≧1×10⁹/L and platelet count ≧100×10⁹/L and normal marrow differential with ≦5% blast cells (Cheson, et al. Blood, 1996; 87: 4990-4997).

As used herein “partial remission” means “complete remission” with 6% to 25% of abnormal cells in the marrow or 50% decrease in bone marrow blasts.

CRp: As per CR but platelet count <100×10⁹/L.

As used herein “morphologic leukemia-free state” means normal morrow differential (<5 blasts); neutorphil and platelet counts are not considered.

As used herein “marrow complete response” means bone marrow 5% myeloblasts and decrease by ≧50% over pre-treatment.

Hematologic Improvement (HI): Hematologic improvement is described by the number of individual, positively affected cell lines (e.g. HI-E; HI-E+HI-N; HI-E+HI-P+HI-N).

### Erythroid Response (HI-E)

Major response: For subjects with pretreatment hemoglobin less than 11 g/dL, greater than 2 g/dL increase in hemoglobin; for RBC transfusion-dependent subjects, transfusion independence.
Minor response: For subjects with pretreatment hemoglobin less than 11 g/dL, 1 to 2 g/dL increase in hemoglobin; for RBC transfusion-dependent subjects, 50% decrease in transfusion requirements.

### Platelet Response (HI-P)

Major response: For subjects with a pretreatment platelet count less than 100×10⁹/L, an absolute increase of 30×10⁹/L or more; for platelet transfusion-dependent subjects, stabilization of platelet transfusion independence.
Minor response: For subjects with a pretreatment platelet count less than 100×10⁹/L a 50% or more increase in platelet count with a net increase greater than 10×10⁹/L but less than 30×10⁹/L.

### Neutrophil Response (HI-N)

Major response: For absolute neutrophil count (ANC) less than 1.5×10⁹/L before therapy, at least a 100% increase, or an absolute increase of more than 0.5×10⁹/L, whichever is greater.
Minor response: For ANC less than 1.5×10⁹/L before therapy, ANC increase of at least 100%, but absolute increase less than 0.5×10⁹/L.

### Progression/Relapse After HI:

One or more of the following: a 50% or greater decrement from maximum response levels in granulocytes or platelets, a reduction in hemoglobin concentration by at least 2 g/dL, or transfusion.

In patients with chronic lymphocytic leukemia (CLL) the following clinical outcome definitions are used (Cheson, et al. Blood, 1996; 87: 4990-4997).

As used herein “complete response” is used in describing subjects having chronic lymphocytic leukemia (CLL) (Cheson, et al. Blood, 1996; 87: 4990-4997) and means:
Peripheral blood—Absolute lymphocyte count (ALC)<4×10⁹/L with Hb>11 g/dl, ANC≧1.5×10⁹/L and platelet count >100×10⁹/L;
Tumor—disappearance of all palpable lymph nodes, spleen and liver without the appearance of new lesions; and
Bone marrow—<30% lymphocytes in normocellular marrow, if lymphoid nodules are seen, response is deemed as nodular CR.

As used herein “partial response” is used in describing subjects having CLL and means:
Peripheral blood—Absolute lymphocyte count (ALC) reduced by 50% from pretreatment baseline value, Hb>11 g/dl or 50% improvement from baseline without transfusion, ANC≧1.5×10⁹/L and platelet count or 50% improvement over baseline, and platelet count >100×10⁹/L or 50% improvement over baseline;
Tumor—When compared with pretreatment measurements, a reduction of ≧50% in measureable lesions without the appearance of new lesions, disappearance of all palpable lymph nodes, spleen and liver without the appearance of new lesions; and

Bone marrow—<30% lymphocytes in normocellular marrow, if lymphoid nodules are seen, response is deemed as nodular CR.

As used herein “Stable disease (SD)” means no CR or PR, no progressive disease.

As used herein “Progressive Disease (PD)” or “Relapse of Disease” is used in describing subjects having CLL and means
Peripheral blood: A ≧50% increase in ALC over baseline in first course, or lowest prior thereafter, with a sustained level >10×10⁹/L.
Tumor: An increase in the product of two perpendicular diameters of a measured lesion by 50% over the size present at entry on study or for subjects who respond, the size at the time of maximum regression and/or the appearance of new areas of malignant disease. A deterioration in performance status or increasing symptoms do not constitute progression; however, their appearance should initiate a new evaluation for extent of disease.

For the disease Agnogenic Myeloid Metaplasia (AMM) the following clinical response definitions apply (Tefferi, et al. Blood. 2007; 110: 1092-1097)

Complete Response: Absence of signs or symptoms of the disease. WBC between 1 to 10×10⁹/L with no peripheral blasts, promyelocytes, or myelocytes and with normalization of bone marrow (<5% blasts in normocellular or hypercellular marrow).

### Resolution of Pretreatment Cytopenias:

ANC≧1.0×10⁹/L without G-CSF or GM-CSF
   Hgb≧12.0 gm/dl (11.0 gm/dl for females) without erythropoietin or transfusion support.
PLT≧100×10⁹/L without growth factor or transfusion support.
   Resolution of Pretreatment Leukocytosis and/or Thrombocytosis:
WBC≦10×10⁹/L without peripheral blasts, promyelocytes, or myelocytes
PLT≦100×10⁹/L but less than 450×10⁹/L

Partial Response:

### Improvement of Two or More of the Following:

ANC: Increase by 100% and to above 10⁹/L for neutropenia
WBC: between 1-10×10⁹/L with persistence of immature cells (blasts, myelocytes, metamyelocytes) for pretreatment leukocytosis.
Hemoglobin: Increase by 2 gm/dL if it was below 10 gm/dL or decrease in transfusion requirements by at least 50% (decrease in frequency and/or volume)
Platelet Count: below that level prior to therapy or persistent thrombocytosis >450×10⁹/L but <50% of pre-treatment
Marrow Blasts Reduction of marrow blasts to 5% or less if it was above 10% in normocellular or hypercellular marrow
Organomegaly: Reduction in splenomegaly and/or hepatomegaly by 50% of pretreatment dimensions (measured as length below the left costal margin on palpation) confirmed by imaging in difficult cases.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

By the term “combination” and grammatical variations thereof, as used herein is meant either simultaneous administration or any manner of separate sequential administration of a therapeutically effective amount of Compound A, or a pharmaceutically acceptable salt or solvate thereof, and Compound B or a pharmaceutically acceptable salt thereof. Preferably, if the administration is not simultaneous, the compounds are administered in a close time proximity to each other. Furthermore, it does not matter if the compounds are administered in the same dosage form, e.g. one compound may be administered topically and the other compound may be administered orally. Suitably, both compounds are administered orally.

By the term “combination kit” as used herein is meant the pharmaceutical composition or compositions that are used to administer Compound A, or a pharmaceutically acceptable salt or solvate thereof, and Compound B, or a pharmaceutically acceptable salt thereof, according to the invention. When both compounds are administered simultaneously, the combination kit can contain Compound A, or a pharmaceutically acceptable salt or solvate thereof, and Compound B, or a pharmaceutically acceptable salt thereof, in a single pharmaceutical composition, such as a tablet, or in separate pharmaceutical compositions. When the compounds are not administered simultaneously, the combination kit will contain Compound A, or a pharmaceutically acceptable salt or solvate thereof, and Compound B, or a pharmaceutically acceptable salt thereof, in separate pharmaceutical compositions. The combination kit can comprise Compound A, or a pharmaceutically acceptable salt or solvate thereof, and Compound B, or a pharmaceutically acceptable salt thereof, in separate pharmaceutical compositions in a single package or in separate pharmaceutical compositions in separate packages.

In one aspect there is provided a combination kit comprising the components:
Compound A, or a pharmaceutically acceptable salt or solvate thereof, in association with a pharmaceutically acceptable carrier; and
Compound B, or a pharmaceutically acceptable salt thereof, in association with a pharmaceutically acceptable carrier.

In one embodiment of the invention the combination kit comprises the following components:
Compound A, or a pharmaceutically acceptable salt or solvate thereof, in association with a pharmaceutically acceptable carrier; and
Compound B, or a pharmaceutically acceptable salt thereof, in association with a pharmaceutically acceptable carrier,
   wherein the components are provided in a form which is suitable for sequential, separate and/or simultaneous administration.

In one embodiment the combination kit comprises:
a first container comprising Compound A, or a pharmaceutically acceptable salt or solvate thereof, in association with a pharmaceutically acceptable carrier; and
a second container comprising Compound B, or a pharmaceutically acceptable salt thereof, in association with a pharmaceutically acceptable carrier, and a container means for containing said first and second containers.

The “combination kit” can also be provided by instruction, such as dosage and administration instructions. Such dosage and administration instructions can be of the kind that is provided to a doctor, for example by a drug product label, or they can be of the kind that is provided by a doctor, such as instructions to a patient.

As used herein the term “Compound A²” means—Compound A, or a pharmaceutically acceptable salt or solvate thereof—.

As used herein the term “Compound B²” means—Compound B, or a pharmaceutically acceptable salt thereof—.

Suitably the combinations of this invention are administered within a “specified period”.

By the term “specified period” and grammatical variations thereof, as used herein is meant the interval of time between the administration of one of Compound A² and Compound B² and the other of Compound A² and Compound B². Unless otherwise defined, the specified period can include simultaneous administration. Unless otherwise defined the specified period refers to administration of Compound A² and Compound B² during a single day.

Suitably, if the compounds are administered within a “specified period” and not administered simultaneously, they are both administered within about 24 hours of each other—in this case, the specified period will be about 24 hours; suitably they will both be administered within about 12 hours of each other—in this case, the specified period will be about 12 hours; suitably they will both be administered within about 11 hours of each other—in this case, the specified period will be about 11 hours; suitably they will both be administered within about 10 hours of each other—in this case, the specified period will be about 10 hours; suitably they will both be administered within about 9 hours of each other—in this case, the specified period will be about 9 hours; suitably they will both be administered within about 8 hours of each other—in this case, the specified period will be about 8 hours; suitably they will both be administered within about 7 hours of each other—in this case, the specified period will be about 7 hours; suitably they will both be administered within about 6 hours of each other—in this case, the specified period will be about 6 hours; suitably they will both be administered within about 5 hours of each other—in this case, the specified period will be about 5 hours; suitably they will both be administered within about 4 hours of each other—in this case, the specified period will be about 4 hours; suitably they will both be administered within about 3 hours of each other—in this case, the specified period will be about 3 hours; suitably they will be administered within about 2 hours of each other—in this case, the specified period will be about 2 hours; suitably they will both be administered within about 1 hour of each other—in this case, the specified period will be about 1 hour. As used herein, the administration of Compound A² and Compound B² in less than about 45 minutes apart is considered simultaneous administration.

Suitably, when the combination of the invention is administered for a “specified period”, the compounds will be co-administered for a “duration of time”.

By the term “duration of time” and grammatical variations thereof, as used herein is meant that both compounds of the invention are administered for an indicated number of consecutive days. Unless otherwise defined, the number of consecutive days does not have to commence with the start of treatment or terminate with the end of treatment, it is only required that the number of consecutive days occur at some point during the course of treatment.

### Regarding “Specified Period” Administration:

Suitably, both compounds will be administered within a specified period for at least one day—in this case, the duration of time will be at least one day; suitably, during the course to treatment, both compounds will be administered within a specified period for at least 3 consecutive days—in this case, the duration of time will be at least 3 days; suitably, during the course to treatment, both compounds will be administered within a specified period for at least 5 consecutive days—in this case, the duration of time will be at least 5 days; suitably, during the course to treatment, both compounds will be administered within a specified period for at least 7 consecutive days—in this case, the duration of time will be at least 7 days; suitably, during the course to treatment, both compounds will be administered within a specified period for at least 14 consecutive days—in this case, the duration of time will be at least 14 days; suitably, during the course to treatment, both compounds will be administered within a specified period for at least 30 consecutive days—in this case, the duration of time will be at least 30 days.

Suitably, if the compounds are not administered during a “specified period”, they are administered sequentially. By the term “sequential administration”, and derivates thereof, as used herein is meant that one of Compound A² and Compound B² is administered once a day for two or more consecutive days and the other of Compound A² and Compound B² is subsequently administered once a day for two or more consecutive days. Also, contemplated herein is a drug holiday utilized between the sequential administration of one of Compound A² and Compound B² and the other of Compound A² and Compound B². As used herein, a drug holiday is a period of days after the sequential administration of one of Compound A² and Compound B² and before the administration of the other of Compound A² and Compound B² where neither Compound A² nor Compound B² is administered. Suitably the drug holiday will be a period of days selected from: 1 day, 2 days, 3 days, 4 days, 5 days, 6 days, 7 days, 8 days, 9 days, 10 days, 11 days, 12 days, 13 days and 14 days.

### Regarding Sequential Administration:

Suitably, one of Compound A² and Compound B² is administered for from 2 to 30 consecutive days, followed by an optional drug holiday, followed by administration of the other of Compound A² and Compound B² for from 2 to 30 consecutive days. Suitably, one of Compound A² and Compound B² is administered for from 2 to 21 consecutive days, followed by an optional drug holiday, followed by administration of the other of Compound A² and Compound B² for from 2 to 21 consecutive days. Suitably, one of Compound A² and Compound B² is administered for from 2 to 14 consecutive days, followed by a drug holiday of from 1 to 14 days, followed by administration of the other of Compound A² and Compound B² for from 2 to 14 consecutive days. Suitably, one of Compound A² and Compound B² is administered for from 3 to 7 consecutive days, followed by a drug holiday of from 3 to 10 days, followed by administration of the other of Compound A² and Compound B² for from 3 to 7 consecutive days.

Suitably, Compound B² will be administered first in the sequence, followed by an optional drug holiday, followed by administration of Compound A². Suitably, Compound B² is administered for from 3 to 21 consecutive days, followed by an optional drug holiday, followed by administration of Compound A² for from 3 to 21 consecutive days. Suitably, Compound B² is administered for from 3 to 21 consecutive days, followed by a drug holiday of from 1 to 14 days, followed by administration of Compound A² for from 3 to 21 consecutive days. Suitably, Compound B² is administered for from 3 to 21 consecutive days, followed by a drug holiday of from 3 to 14 days, followed by administration of Compound A² for from 3 to 21 consecutive days. Suitably, Compound B² is administered for 21 consecutive days, followed by an optional drug holiday, followed by administration of Compound A² for 14 consecutive days. Suitably, Compound B² is administered for 14 consecutive days, followed by a drug holiday of from 1 to 14 days, followed by administration of Compound A² for 14 consecutive days. Suitably, Compound B² is administered for 7 consecutive days, followed by a drug holiday of from 3 to 10 days, followed by administration of Compound A² for 7 consecutive days. Suitably, Compound B² is administered for 3 consecutive days, followed by a drug holiday of from 3 to 14 days, followed by administration of Compound A² for 7 consecutive days. Suitably, Compound B² is administered for 3 consecutive days, followed by a drug holiday of from 3 to 10 days, followed by administration of Compound A² for 3 consecutive days.

It is understood that a “specified period” administration and a “sequential” administration can be followed by repeat dosing or can be followed by an alternate dosing protocol, and a drug holiday may precede the repeat dosing or alternate dosing protocol.

Suitably, the amount of Compound A² administered as part of the combination according to the present invention will be an amount selected from about 0.125 mg to about 10 mg; suitably, the amount will be selected from about 0.25 mg to about 9 mg; suitably, the amount will be selected from about 0.25 mg to about 8 mg; suitably, the amount will be selected from about 0.5 mg to about 8 mg; suitably, the amount will be selected from about 0.5 mg to about 7 mg; suitably, the amount will be selected from about 1 mg to about 7 mg; suitably, the amount will be about 5 mg. Accordingly, the amount of Compound A administered as part of the combination according to the present invention will be an amount selected from about 0.125 mg to about 10 mg. For example, the amount of Compound A² administered as part of the combination according to the present invention can be 0.125 mg, 0.25 mg, 0.5 mg, 0.75 mg, 1 mg, 1.5 mg, 2 mg, 2.5 mg, 3 mg, 3.5 mg, 4 mg, 4.5 mg, 5 mg, 5.5 mg, 6 mg, 6.5 mg, 7 mg, 7.5 mg, 8 mg, 8.5 mg, 9 mg, 9.5 mg, 10 mg.

Suitably, the amount of Compound B² administered as part of the combination according to the present invention will be an amount selected from about 75 mg to about 1,000 mg; suitably, the amount will be selected from about 100 mg to about 900 mg; suitably, the amount will be selected from about 150 mg to about 850 mg; suitably, the amount will be selected from about 200 mg to about 800 mg; suitably, the amount will be selected from about 250 mg to about 750 mg; suitably, the amount will be selected from about 300 mg to about 6000 mg; suitably, the amount will be about 450 mg. Accordingly, the amount of Compound B² administered as part of the combination according to the present invention will be an amount selected from about 75 mg to about 1,000 mg. For example, the amount of Compound B² administered as part of the combination according to the present invention can be 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 550 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg or 1,000 mg.

As used herein, all amounts specified for Compound A² and Compound B² are indicated as the administered amount of free or unsalted and unsolvated compound per dose.

The method of the present invention may also be employed with other therapeutic methods of cancer treatment.

While it is possible that, for use in therapy, therapeutically effective amounts of the combinations of the present invention may be administered as the raw chemical, it is preferable to present the combinations as a pharmaceutical composition or compositions. Accordingly, the invention further provides pharmaceutical compositions, which include Compound A² and/or Compound B², and one or more pharmaceutically acceptable carriers. The combinations of the present invention are as described above. The carrier(s) must be acceptable in the sense of being compatible with the other ingredients of the formulation, capable of pharmaceutical formulation, and not deleterious to the recipient thereof. In accordance with another aspect of the invention there is also provided a process for the preparation of a pharmaceutical formulation including admixing Compound A² and/or Compound B² with one or more pharmaceutically acceptable carriers. As indicated above, such elements of the pharmaceutical combination utilized may be presented in separate pharmaceutical compositions or formulated together in one pharmaceutical formulation.

Pharmaceutical formulations may be presented in unit dose forms containing a predetermined amount of active ingredient per unit dose. As is known to those skilled in the art, the amount of active ingredient per dose will depend on the condition being treated, the route of administration and the age, weight and condition of the patient. Preferred unit dosage formulations are those containing a daily dose or sub-dose, or an appropriate fraction thereof, of an active ingredient. Furthermore, such pharmaceutical formulations may be prepared by any of the methods well known in the pharmacy art.

Compound A² and Compound B² may be administered by any appropriate route. Suitable routes include oral, rectal, nasal, topical (including buccal and sublingual), vaginal, and parenteral (including subcutaneous, intramuscular, intravenous, intradermal, intrathecal, and epidural). It will be appreciated that the preferred route may vary with, for example, the condition of the recipient of the combination and the cancer to be treated. It will also be appreciated that each of the agents administered may be administered by the same or different routes and that Compound A² and Compound B² may be compounded together in a pharmaceutical composition/formulation.

The compounds or combinations of the current invention are incorporated into convenient dosage forms such as capsules, tablets, or injectable preparations. Solid or liquid pharmaceutical carriers are employed. Solid carriers include, starch, lactose, calcium sulfate dihydrate, terra alba, sucrose, talc, gelatin, agar, pectin, acacia, magnesium stearate, and stearic acid. Liquid carriers include syrup, peanut oil, olive oil, saline, and water. Similarly, the carrier may include a prolonged release material, such as glyceryl monostearate or glyceryl distearate, alone or with a wax. The amount of solid carrier varies widely but, preferably, will be from about 25 mg to about 1 g per dosage unit. When a liquid carrier is used, the preparation will suitably be in the form of a syrup, elixir, emulsion, soft gelatin capsule, sterile injectable liquid such as an ampoule, or an aqueous or nonaqueous liquid suspension.

For instance, for oral administration in the form of a tablet or capsule, the active drug component can be combined with an oral, non-toxic pharmaceutically acceptable inert carrier such as ethanol, glycerol, water and the like. Powders are prepared by comminuting the compound to a suitable fine size and mixing with a similarly comminuted pharmaceutical carrier such as an edible carbohydrate, as, for example, starch or mannitol. Flavoring, preservative, dispersing and coloring agent can also be present.

It should be understood that in addition to the ingredients mentioned above, the formulations may include other agents conventional in the art having regard to the type of formulation in question, for example those suitable for oral administration may include flavoring agents.

As indicated, therapeutically effective amounts of the combinations of the invention (Compound A² in combination with Compound B²) are administered to a human. Typically, the therapeutically effective amount of the administered agents of the present invention will depend upon a number of factors including, for example, the age and weight of the subject, the precise condition requiring treatment, the severity of the condition, the nature of the formulation, and the route of administration. Ultimately, the therapeutically effective amount will be at the discretion of the attendant physician.

The combinations of the present invention are tested for efficacy, advantageous and synergistic properties according to known procedures. Suitably, the combinations of the invention are tested for efficacy, advantageous and synergistic properties generally according to the following combination cell proliferation assays. Cells are plated in 384-well plates at 500 cells/well in culture media appropriate for each cell type, supplemented with 10% FBS and 1% penicillin/streptomycin, and incubated overnight at 37° C., 5% CO₂. Cells are treated in a grid manner with dilution of Compound A² (20 dilutions, including no compound, of 2-fold dilutions starting from 1-20 μM depending of compound) from left to right on 384-well plate and also treated with Compound B² (20 dilutions, including no compound, of 2-fold dilutions starting from 1-20 μM depending of compound) from top to bottom on 384-well plate and incubated as above for a further 72 hours. In some instances compounds are added in a staggered manner and incubation time can be extended up to 7 days. Cell growth is measured using CellTiter-Glo® reagent according to the manufacturer's protocol and signals are read on a PerkinElmer EnVision™ reader set for luminescence mode with a 0.5-second read. Data are analyzed as described below.

Results are expressed as a percentage of the t=0 value and plotted against compound(s) concentration. The t=0 value is normalized to 100% and represents the number of cells present at the time of compound addition. The cellular response is determined for each compound and/or compound combination using a 4- or 6-parameter curve fit of cell viability against concentration using the IDBS XLfit plug-in for Microsoft Excel software and determining the concentration required for 50% inhibition of cell growth (gIC₅₀). Background correction is made by subtraction of values from wells containing no cells. For each drug combination a Combination Index (CI), Excess Over Highest Single Agent (EOHSA) and Excess Over Bliss (EOBliss) are calculated according to known methods such as described in Chou and Talalay (1984) Advances in Enzyme Regulation, 22, 37 to 55; and Berenbaum, M C (1981) Adv. Cancer Research, 35, 269-335.

Because the combinations of the present invention are active in the above assays they exhibit advantageous therapeutic utility in treating cancer.

Suitably, the present invention relates to a method for treating or lessening the severity of a cancer selected from: brain (gliomas), glioblastomas, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone, thyroid,

Lymphoblastic T cell leukemia, Chronic myelogenous leukemia, Chronic lymphocytic leukemia, Hairy-cell leukemia, acute lymphoblastic leukemia, acute myelogenous leukemia, Chronic neutrophilic leukemia, Acute lymphoblastic T cell leukemia, Plasmacytoma, Immunoblastic large cell leukemia, Mantle cell leukemia, Multiple myeloma Megakaryoblastic leukemia, multiple myeloma, acute megakaryocytic leukemia, promyelocytic leukemia, Erythroleukemia,

malignant lymphoma, hodgkins lymphoma, non-hodgkins lymphoma, lymphoblastic T cell lymphoma, Burkitt's lymphoma, follicular lymphoma,

neuroblastoma, bladder cancer, urothelial cancer, lung cancer, vulval cancer, cervical cancer, endometrial cancer, renal cancer, mesothelioma, esophageal cancer, salivary gland cancer, hepatocellular cancer, gastric cancer, nasopharangeal cancer, buccal cancer, cancer of the mouth, GIST (gastrointestinal stromal tumor) and testicular cancer.

Suitably, the present invention relates to a method for treating or lessening the severity of a cancer selected from: brain (gliomas), glioblastomas, Bannayan-Zonana syndrome, Cowden disease, Lhermitte-Duclos disease, breast, colon, head and neck, kidney, lung, liver, melanoma, ovarian, pancreatic, prostate, sarcoma and thyroid.

Suitably, the present invention relates to a method for treating or lessening the severity of a cancer selected from ovarian, breast, pancreatic and prostate.

Suitably the present invention relates to methods for treating or lessening the severity of a cancer selected from leukemia and myeloid malignancy.

As used herein, the terms “cancer,” “neoplasm,” and “tumor,” are used interchangeably and in either the singular or plural form, refer to cells that have undergone a malignant transformation that makes them pathological to the host organism. Primary cancer cells (that is, cells obtained from near the site of malignant transformation) can be readily distinguished from non-cancerous cells by well-established techniques, particularly histological examination. The definition of a cancer cell, as used herein, includes not only a primary cancer cell, but any cell derived from a cancer cell ancestor. This includes metastasized cancer cells, and in vitro cultures and cell lines derived from cancer cells. When referring to a type of cancer that normally manifests as a solid tumor, a “clinically detectable” tumor is one that is detectable on the basis of tumor mass; e.g., by procedures such as CAT scan, MR imaging, X-ray, ultrasound or palpation, and/or which is detectable because of the expression of one or more cancer-specific antigens in a sample obtainable from a patient. Tumors may be hematopoietic tumor, for example, tumors of blood cells or the like, meaning liquid tumors. Specific examples of clinical conditions based on such a tumor include leukemia such as chronic myelocytic leukemia or acute myelocytic leukemia; myeloma such as multiple myeloma; lymphoma and the like.

Typically, any anti-neoplastic agent that has activity versus a susceptible tumor being treated may be co-administered in the treatment of cancer in the present invention. Examples of such agents can be found in Cancer Principles and Practice of Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; receptor tyrosine kinase inhibitors; serine-threonine kinase inhibitors; non-receptor tyrosine kinase inhibitors; angiogenesis inhibitors, immunotherapeutic agents; proapoptotic agents; and cell cycle signaling inhibitors.

The present invention also provides methods for treating cancer comprising administering Compound A or pharmaceutically acceptable salt thereof with or without a Braf inhibitor, including, but not limited to, Compound B or a pharmaceutically acceptable salt or solvate thereof and another anti-neoplastic agent.

Examples of a further active ingredient or ingredients (anti-neoplastic agent) for use in combination or co-administered with Compound A or pharmaceutically acceptable salt thereof are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)-N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem, Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intern, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guide, 1998) related to the duration of dosing above a threshold concentration (50 nM) (Kearns, C. M. et. al., Seminars in Oncology, 3(6) p. 16-23, 1995).

Docetaxel, (2R,3S)-N-carboxy-3-phenylisoserine,N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin Ill, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R-(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthracyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME® or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (6-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl)methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I:DNA:irintecan or SN-38 ternary complex with replication enzymes. Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Rituximab is a chimeric monoclonal antibody which is sold as RITUXAN® and MABTHERA®. Rituximab binds to CD20 on B cells and causes cell apoptosis. Rituximab is administered intravenously and is approved for treatment of rheumatoid arthritis and B-cell non-Hodgkin's lymphoma.

Ofatumumab is a fully human monoclonal antibody which is sold as ARZERRA®. Ofatumumab binds to CD20 on B cells and is used to treat chronic lymphocytic leukemia (CLL; a type of cancer of the white blood cells) in adults who are refractory to treatment with fludarabine (Fludara) and alemtuzumab (Campath).

mTOR inhibitors include but are not limited to rapamycin and rapalogs, RAD001 or everolimus (Afinitor), CCI-779 or temsirolimus, AP23573, AZD8055, WYE-354, WYE-600, WYE-687 and Pp121.

Bexarotene is sold as Targretin® and is a member of a subclass of retinoids that selectively activate retinoid X receptors (RXRs). These retinoid receptors have biologic activity distinct from that of retinoic acid receptors (RARs). The chemical name is 4-[1-(5,6,7,8-tetrahydro-3,5,5,8,8-pentamethyl-2-naphthalenyl)ethenyl]benzoic acid. Bexarotene is used to treat cutaneous T-cell lymphoma (CTCL, a type of skin cancer) in people whose disease could not be treated successfully with at least one other medication.

Sorafenib marketed as Nexavar® is in a class of medications called multikinase inhibitors. Its chemical name is 4-[4-[[4-chloro-3-(trifluoromethyl)phenyl]carbamoylamino]phenoxy]-N-methyl-pyridine-2-carboxamide. Sorafenib is used to treat advanced renal cell carcinoma (a type of cancer that begins in the kidneys). Sorafenib is also used to treat unresectable hepatocellular carcinoma (a type of liver cancer that cannot be treated with surgery).

Suitably, the present invention relates to a method of treating or lessening the severity of a cancer that is either wild type or mutant for Raf and either wild type or mutant for PI3K/Pten. This includes patients wild type for both or either Raf and PI3K/PTEN while also being Ras mutant.

The term “wild type” as is understood in the art refers to a polypeptide or polynucleotide sequence that occurs in a native population without genetic modification. As is also understood in the art, a “mutant” includes a polypeptide or polynucleotide sequence having at least one modification to an amino acid or nucleic acid compared to the corresponding amino acid or nucleic acid found in a wild type polypeptide or polynucleotide, respectively. Included in the term mutant is Single Nucleotide Polymorphism (SNP) where a single base pair distinction exists in the sequence of a nucleic acid strand compared to the most prevalently found (wild type) nucleic acid strand.

As used herein, “genotyping” a cell including a tumor cell from a subject (or DNA or other biological sample) for a mutation or a polymorphic allele of a gene(s) means detecting which allelic or polymorphic form(s) and/or wild type or somatically mutated form(s) of the gene(s) or gene expression products (e.g., hnRNA, mRNA or protein) are present or absent in a subject (or a sample). Related RNA or protein expressed from such gene may also be used to detect polymorphic variation. For purposes of the present invention, “genotyping” includes the determination of somatic as well as genotypic mutations from a sample. As used herein, an allele may be ‘detected’ when other possible allelic variants have been ruled out; e.g., where a specified nucleic acid position is found to be neither adenine (A), thymine (T) or cytosine (C), it can be concluded that guanine (G) is present at that position (i.e., G is ‘detected’ or ‘diagnosed’ in a subject). Sequence variations may be detected directly (by, e.g. sequencing, for example, EST sequencing or partial or full genome sequencing) or indirectly (e.g., by restriction fragment length polymorphism analysis, or detection of the hybridization of a probe of known sequence, or reference strand conformation polymorphism), or by using other known methods.

The sequence of any nucleic acid including a gene or PCR product or a fragment or portion thereof may be sequenced by any method known in the art (e.g., chemical sequencing or enzymatic sequencing). “Chemical sequencing” of DNA may denote methods such as that of Maxam and Gilbert (1977) (Proc. Natl. Acad. Sci. USA 74:560), in which DNA is randomly cleaved using individual base-specific reactions. “Enzymatic sequencing” of DNA may denote methods such as that of Sanger (Sanger, et al., (1977) Proc. Natl. Acad. Sci. USA 74:5463).

Conventional molecular biology, microbiology, and recombinant DNA techniques including sequencing techniques are well known among those skilled in the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook, et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985)); Transcription And Translation (B. D. Hames & S. J. Higgins, eds. (1984)); Animal Cell Culture (R. I. Freshney, ed. (1986)); Immobilized Cells And Enzymes (IRL Press, (1986)); B. Perbal, A Practical Guide To Molecular Cloning (1984); F. M. Ausubel, et al. (eds.), Current Protocols in Molecular Biology, John Wiley & Sons, Inc. (1994

The Peptide Nucleic Acid (PNA) affinity assay is a derivative of traditional hybridization assays (Nielsen et al., Science 254:1497-1500 (1991); Egholm et al., J. Am. Chem. Soc. 114:1895-1897 (1992); James et al., Protein Science 3:1347-1350 (1994)). PNAs are structural DNA mimics that follow Watson-Crick base pairing rules, and are used in standard DNA hybridization assays. PNAs display greater specificity in hybridization assays because a PNA/DNA mismatch is more destabilizing than a DNA/DNA mismatch and complementary PNA/DNA strands form stronger bonds than complementary DNA/DNA strands.

DNA microarrays have been developed to detect genetic variations and polymorphisms (Taton et al., Science 289:1757-60, 2000; Lockhart et al., Nature 405:827-836 (2000); Gerhold et al., Trends in Biochemical Sciences 24:168-73 (1999); Wallace, R. W., Molecular Medicine Today 3:384-89 (1997); Blanchard and Hood, Nature Biotechnology 149:1649 (1996)). DNA microarrays are fabricated by high-speed robotics, on glass or nylon substrates, and contain DNA fragments with known identities (“the probe”). The microarrays are used for matching known and unknown DNA fragments (“the target”) based on traditional base-pairing rules.

The term “at least one mutation” in a polypeptide or a gene encoding a polypeptide and grammatical variations thereof means a polypeptide or gene encoding a polypeptide having one or more allelic variants, splice variants, derivative variants, substitution variants, deletion variants, truncation variants, and/or insertion variants, fusion polypeptides, orthologs, and/or interspecies homologs. By way of example, at least one mutation of a Ras protein would include a Ras protein in which part of all of the sequence of a polypeptide or gene encoding the Ras protein is absent or not expressed in the cell for at least one Ras protein produced in the cell. For example, a Ras protein may be produced by a cell in a truncated form and the sequence of the truncated form may be wild type over the sequence of the truncate. A deletion may mean the absence of all or part of a gene or protein encoded by a gene. Additionally, some of a protein expressed in or encoded by a cell may be mutated while other copies of the same protein produced in the same cell may be wild type. By way of another example a mutation in a Ras protein would include a Ras protein having one or more amino acid differences in its amino acid sequence compared with wild type of the same Ras protein.

As used herein “genetic abnormality” is meant a deletion, substitution, addition, translocation, amplification and the like relative to the normal native nucleic acid content of a cell of a subject.

The terms “polypeptide” and “protein” are used interchangeably and are used herein as a generic term to refer to native protein, fragments, peptides, or analogs of a polypeptide sequence. Hence, native protein, fragments, and analogs are species of the polypeptide genus.

The terminology “X#Y” in the context of a mutation in a polypeptide sequence is art-recognized, where “#” indicates the location of the mutation in terms of the amino acid number of the polypeptide, “X” indicates the amino acid found at that position in the wild-type amino acid sequence, and “Y” indicates the mutant amino acid at that position. For example, the notation “G12S” with reference to the K-ras polypeptide indicates that there is a glycine at amino acid number 12 of the wild-type K-ras sequence, and that glycine is replaced with a serine in the mutant K-ras sequence.

The term “Ras protein” as used herein means any protein which is a member of the ras subfamily which is a subfamily of GTPases involved in cellular signaling. As is known in the art, activation of Ras causes cell growth, differentiation and survival. Ras proteins include, but are not limited to, H-ras, K-ras and N-ras.

As used herein “gene encoding a Ras protein” means any part of a gene or polynucleotide encoding any Ras protein. Included within the meaning of this term are exons encoding Ras. Genes encoding Ras proteins include but are not limited to genes encoding part or all of H-ras, K-ras and N-ras.

The frequency of patients with acute myeloid leukemia and with at least one RAS mutation is about 12% to about 15% (about 5% KRAS and about 7% to about 10% NRAS). There are about 10-19 different RAS mutant variants which account for about 80-100% of all mutations in RAS mutant tumors. Not all KRAS mutational events are consistent with other RAS mut+ tumors. For example, the frequency of certain Ras mutations vary among tumor types eg, AML, NSCLC, CRC, and pancreatic.

In some tumor types Ras mutations may be mutually exclusive from other RAS mutations and other genetic mutations. For instance, in NSCLS tumors EGFR mutations and K-RAS mutations are mutually exclusive.

Additionally in AML, N-RAS and K-RAS mutations are mutually exclusive. Thus, a tumor cell may have one or more mutations in N-RAS and/or K-RAS. RAS mutation status correlates with FAB cytogenetic classification. RAS mutations are overrepresented in AML M2 and M4 group. RAS/FLT3 mutations are mutually exclusive. N-RAS mutations are underrepresented in FAB M3 indication (promyelocytic leukemia) with t(15;17), where FLT3 ITD is overrepresented (Bowen et al., Blood (2005) 106(6):2133-2119).

A summary of the estimated frequency of N-Ras and K-Ras mutations in AML tumors is summarized in Table 1.

TABLE 1
Frequency of N-RAS and K-RAS mutations in AML

| | | | Nucleotide | Nucleotide | Codon | Codon | Frequency | Percentage |
|---|---|---|---|---|---|---|---|---|
| Gene | Mutation | Exon | Position | Change | Position | Change | Percentage | Coverage |
| N-RAS | G12D | 2 | 35 | G > A | 12 | G > D | 47.82% | 47.82% |
| (7%) | Q61R | 3 | 182 | A > G | 61 | Q > R | 13.04% | 60.86% |
| | Q61K | 3 | 181 | C > A | 61 | Q > K | 8.69% | 69.55% |
| | G12S | 2 | 34 | G > A | 12 | G > S | 4.34% | 73.89% |
| | G12A | 2 | 35 | G > C | 12 | G > A | 4.34% | 78.23% |
| | G12V | 2 | 35 | G > T | 12 | G > V | 4.34% | 82.57% |
| | *Q61H | 3 | 183 | A > T | 61 | Q > H | 4.34% | 86.91% |
| | Q61H | 3 | 183 | A > C | 61 | Q > H | | |
| | G13D | 2 | 38 | G > A | 13 | G > D | 4.34% | 91% |
| | G13R | 2 | 37 | G > C | 13 | G > R | 4.34% | 95.34% |
| | G60E | 3 | 179 | G > A | 60 | G > E | 4.34% | 99.68% |
| K-RAS | G12D | 2 | 35 | G > A | 12 | G > D | 40% | 40% |
| (3%) | G13D | 2 | 38 | G > A | 13 | G > D | 20% | 60% |
| | A146T | 3 | 436 | G > A | 146 | A > T | 20% | 80% |
| | G12A | 2 | 35 | G > C | 12 | G > A | 10% | 90% |
| | V14I | 2 | 40 | G > A | 14 | V > I | 10% | 100% |

Data source: Tyner et al., Blood (2009) 113(80): 1749-1755
N-RAS mutations detected: 23/329 (7%)
K-RAS mutations detected: 9/329 (3%)

A summary of the estimated frequency of association of RAS mutations with different AML cytogenetic groups is presented in Table 2.

TABLE 2
Frequency of association of RAS mutations
with different AML cytogenetic groups

| FAB | N-Ras Mutation | K-Ras Mutation |
|---|---|---|
| Classification | Percentage | Percentage |
| AML M0 | 3% | 5% |
| AML M1 | 16% | 0% |
| AML M2 | 25% | 24% |
| AML M3 | 9% | 13% |
| AML M4 | 30% | 50% |
| AML M5 | 14% | 5% |
| AML M6 | 2% | 0% |
| AML M7 | 0% | 0% |
| RAEB | 2% | 1% |
| Total | 100% | 100% |

Bowen et al., Blood 2005

A summary of the estimated frequency of H-Ras, N-Ras and K-Ras mutations in Non-Small Cell Lung Carcinoma (NSCLC) tumors is summarized in Table 3.

TABLE 3
Frequency of K-Ras, H-Ras and N-Ras mutations in NSCLC

| | | | Nucleotide | Nucleotide | Codon | Codon | Frequency | Percentage |
|---|---|---|---|---|---|---|---|---|
| Gene | Mutation | Exon | Position | Change | Position | Change | Percentage | Coverage |
| K-Ras | G12V | 2 | 35 | G > T | 12 | G > V | 24.72% | 59.15% |
| | G12D | 2 | 35 | G > A | 12 | G > D | 14.12% | 73.27% |
| | G12A | 2 | 35 | G > C | 12 | G > A | 10.15% | 83.42% |
| | G12S | 2 | 34 | G > A | 12 | G > S | 6.18% | 89.6% |
| | G13C | 2 | 37 | G > T | 13 | G > C | 3.31% | 92.91% |
| | G12R | 2 | 34 | G > C | 12 | G > R | 3.09% | 96% |
| | G13D | 2 | 38 | G > A | 12 | G > D | 2.42% | 98.42% |
| | Q61H | 3 | 183 | A > T | 61 | Q > H | 1.32% | 99.74% |
| | Q61L | 3 | 182 | A > T | 61 | Q > L | 0.22% | 99.96% |
| H-Ras | Q61L | 3 | 182 | A > T | 61 | Q > L | Detected in 8/1680 | |
| | | | | | | | samples | |
| | G12C | 2 | 34 | G > T | 12 | G > C | Detected in 1/1680 | |
| | | | | | | | samples | |
| N-Ras | Q61R | 3 | 182 | A > G | 61 | Q > R | 18.18% | 54.78% |
| | Q61L | 3 | 182 | A > T | 61 | Q > L | 9.09% | 63.87% |
| | S65C | 3 | 193 | A > T | 65 | S > C | 9.09% | 72.96% |
| | S65R | 3 | ? | ? | 65 | S > R | 4.54% | 77.5% |
| | G12C | 2 | 34 | G > T | 12 | G > C | 4.54% | 82.04% |
| | Q61E | 3 | 181 | C > G | 61 | Q > E | 4.54% | 86.58% |
| | Q61H | 3 | 183 | A > T | 61 | Q > H | 4.54% | 91.12% |
| | G12D | 2 | 35 | G > A | 12 | G > D | 4.54% | 95.66% |
| | G12A | 2 | 35 | G > C | 12 | G > A | 4.54% | 100% |

A summary of the estimated frequency of H-Ras, N-Ras and K-Ras mutations in Pancreatic Cancer (ductal carcinoma) tumors is summarized in Table 4.

TABLE 4
Frequency of K-RAS mutations in Pancreatic cancer (ductal carcinoma)

| | | | Nucleotide | Nucleotide | Codon | Codon | Frequency | Percentage |
|---|---|---|---|---|---|---|---|---|
| Gene | Mutation | Exon | Position | Change | Position | Change | Percentage | Coverage |
| K-ras | G12V | 2 | 35 | G > T | 12 | G > V | 29.52% | 80.59% |
| | G12R | 2 | 34 | G > C | 12 | G > R | 11.08% | 91.67% |
| | G12C | 2 | 34 | G > T | 12 | G > C | 3.53% | 95.20% |
| | G12A | 2 | 35 | G > C | 12 | G > A | 2.84% | 98.04% |
| | G12S | 2 | 34 | G > A | 12 | G > S | 0.76% | 98.80% |
| | G13D | 2 | 38 | G > A | 13 | G > D | 0.55% | 99.35% |
| | Q61H | 3 | 183 | A > T | 61 | Q > H | 0.34% | 99.69% |

G13C and G12F mutations are negligible.
Data source: COSMIC database, Sanger Institute (http://www.sanger.ac.uk/cosmic)
K-RAS mutations detected: 2429/3532 patient samples (69%)
H-RAS and N-RAS mutations negligible

A summary of the estimated frequency of K-Ras mutations in colorectal tumors is summarized in Table 5.

TABLE 5
Frequency of K-RAS mutations in Colorectal Cancer

| | | | Nucleotide | Nucleotide | Codon | Codon | Frequency | Percentage |
|---|---|---|---|---|---|---|---|---|
| Gene | Mutation | Exon | Position | Change | Position | Change | Percentage | Coverage |
| K-Ras | G12V | 2 | 35 | G > T | 12 | G > V | 25.06% | 54.63% |
| | G13D | 2 | 38 | G > A | 13 | G > D | 24.81% | 79.44% |
| | G12C | 2 | 34 | G > T | 12 | G > C | 7.68% | 87.12% |
| | G12A | 2 | 35 | G > C | 12 | G > A | 6.59% | 93.71% |
| | G12S | 2 | 34 | G > A | 12 | G > S | 6.26% | 99.97% |

Data source: Andreyev et al., Br J cancer, (2001) 85(5): 692-696.
K-RAS mutations detected: 1197/3439 patient samples (34.80%)

The terms “mutant K-ras protein” and “mutant N-ras protein” and “K-ras mutation” and “N-ras mutation” refer to K-ras and N-ras proteins having at least one mutation, respectively. In certain embodiments, Ras mutations include G12S, G12V, G12D, G12A, G12C, G12R, G12S, G13R, G13A, G13D, G13F, G13C, V14I, G60E, Q61H, Q61L, Q61R, Q61E, Q61H, Q61K, S65C, S65R, T74P, E76G, E76K, E76Q, and A146T. Certain N-ras mutations include, but are not limited to G12S, G12V, G12D, G12A, G12C, G12R, G13A, G13D, G13R, G60E, Q61K, Q61H and Q61R. Certain K-ras mutations can occur at positions 12, 13, 59, 61, and 146 and include, but are not limited, to, G12S, G12V, G12D, G12A, G12C, G12R, G13A, G13D, V14I, Q61H, Q61K, Q61R, E76G, E76K, E76Q, and A146T. Ras protein mutation may occur at amino acid 12, 13, 14, 59, 60, 61, 65, 76 and/or 146. Certain exemplary mutant K-ras and N-ras polypeptides include, but are not limited to, allelic variants, splice variants, derivative variants, substitution variants, deletion variants, and/or insertion variants, fusion polypeptides, orthologs, and interspecies homologs. In certain embodiments, a mutant K-ras and N-ras polypeptides includes additional residues at the C- or N-terminus, such as, but not limited to, leader sequence residues, targeting residues, amino terminal methionine residues, lysine residues, tag residues and/or fusion protein residues.

Additionally, mutant Ras polypeptides include polypeptides or gene encoding a polypeptide in which part of all of the polypeptide or gene encoding the polypeptide is deleted or absent from the cell. For example, a Ras protein may be produced by a cell in a truncated form. A deletion may mean the absence of all or part of a gene or protein encoded by a gene.

As used herein the term “amplification” and grammatical variations thereof refers to the presence of one or more extra gene copies in a chromosome complement. In certain embodiments a gene encoding a Ras protein may be amplified in a cell. Amplification of the HER2 gene has been correlated with certain types of cancer. Amplification of the HER2 gene has been found in human salivary gland and gastric tumor-derived cell lines, gastric and colon adenocarcinomas, and mammary gland adenocarcinomas. Semba et al., Proc. Natl. Acad. Sci. USA, 82:6497-6501 (1985); Yokota et al., Oncogene, 2:283-287 (1988); Zhou et al., Cancer Res., 47:6123-6125 (1987); King et al., Science, 229:974-976 (1985); Kraus et al., EMBO J., 6:605-610 (1987); van de Vijver et al., Mol. Cell. Biol., 7:2019-2023 (1987); Yamamoto et al., Nature, 319:230-234 (1986).

As used herein “overexpressed” and “overexpression” of a protein or polypeptide and grammatical variations thereof means that a given cell produces an increased number of a certain protein relative to a normal cell. By way of example, a ras protein may be overexpressed by a tumor cell relative to a non-tumor cell. Additionally, a mutant ras protein may be overexpressed compared to wild type ras protein in a cell. As is understood in the art, expression levels of a polypeptide in a cell can be normalized to a housekeeping gene such as actin. In some instances, a certain polypeptide may be underexpressed in a tumor cell compared with a non-tumor cell.

As used herein “nucleic acid necessary for expression of at least one gene product” refers to a nucleic acid sequence that encodes any portion of a gene and/or is operably linked to a nucleic acid encoding a gene product but does not necessarily comprise encoding sequence. By way of example, a nucleic acid sequence necessary for the expression of at least one gene product includes, but is not limited to, enhancers, promoters, regulatory sequences, start codons, stop codons, polyadenylation sequences, and/or encoding sequences. Expression levels of a polypeptide in a particular cell can be effected by, but not limited to, mutations, deletions and/or substitutions of various regulatory elements and/or non-encoding sequence in the cell genome.

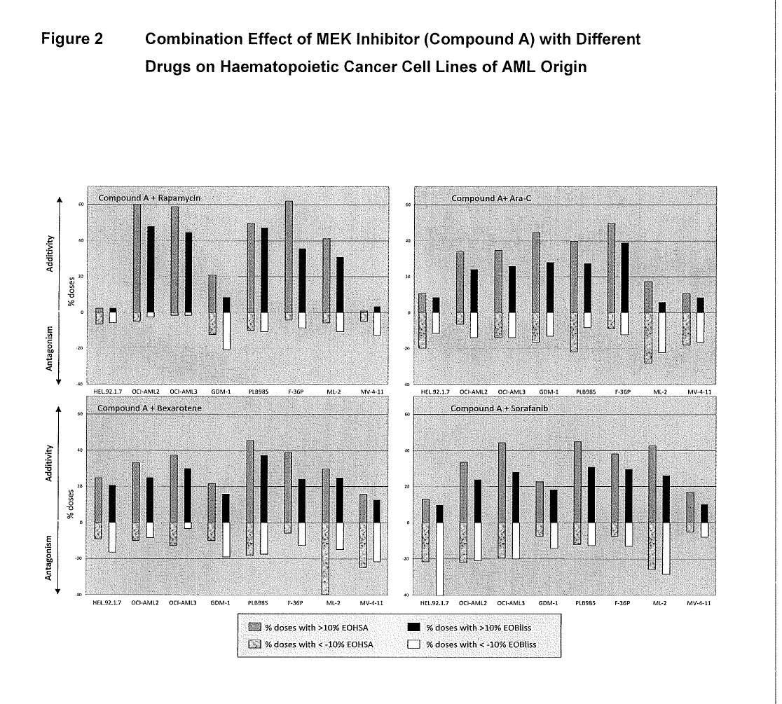

The terms “mutant B-raf protein” refers to a B-raf polypeptide comprising at least one mutation. Certain exemplary mutant B-raf polypeptides include, but are not limited to, allelic variants, splice variants, derivative variants, substitution variants, deletion variants, and/or insertion variants, fusion polypeptides, orthologs, and interspecies homologs. In certain embodiments, a mutant B-raf polypeptide includes additional residues at the C- or N-terminus, such as, but not limited to, leader sequence residues, targeting residues, amino terminal methionine residues, lysine residues, tag residues and/or fusion protein residues. Certain B-raf mutants include but are not limited to BRAF having an amino acid substitution selected from the group consisting of R462I, I463S, G464V, G464E, G466A, G466E, G466V, G469A, G469E, D594V, F595L, G596R, L597V, L597R, T599I, V600E, V600D, V600K, V600R, T119S, and K601E. See, for example, FIG. 2 of Halilovic and Solvit (2008) Current Opinion in Pharmacology 8:419-26. BRAF encodes a RAS-regulated kinase that mediate cell growth and malignant transformation kinase pathway activation.

The term “polynucleotide” as referred to herein means a polymeric form of nucleotides of at least 10 bases in length, either ribonucleotides or deoxynucleotides or a modified form of either type of nucleotide. The term includes single and double stranded forms of DNA.

The term “oligonucleotide” referred to herein includes naturally occurring and modified nucleotides linked together by naturally occurring, and non-naturally occurring oligonucleotide linkages. Oligonucleotides are a polynucleotide subset generally comprising a length of 200 bases or fewer. Preferably oligonucleotides are 10 to 60 bases in length and most preferably 12, 13, 14, 15, 16, 17, 18, 19, or 20 to 40 bases in length. Oligonucleotides are usually single stranded, e.g. for probes, although oligonucleotides may be double stranded, e.g. for use in the construction of a gene mutant. Oligonucleotides can be either sense or antisense oligonucleotides.

An oligonucleotide probe, or probe, is a nucleic acid molecule which typically ranges in size from about 8 nucleotides to several hundred nucleotides in length. Such a molecule is typically used to identify a target nucleic acid sequence in a sample by hybridizing to such target nucleic acid sequence under stringent hybridization conditions. Hybridization conditions have been described in detail above.

PCR primers are also nucleic acid sequences, although PCR primers are typically oligonucleotides of fairly short length which are used in polymerase chain reactions. PCR primers and hybridization probes can readily be developed and produced by those of skill in the art, using sequence information from the target sequence. (See, for example, Sambrook et al., supra or Glick et al., supra).

As is known in the art, several primers are known for use in PCR for detecting Ras and Braf mutations. For example, primers for detecting mutations in Braf and K-ras are presented in several research articles and US patents including, but not limited to, Brose, et al. Cancer Research 62:6997-7000 (2002), Xu, et al. Cancer research 63:4561-4567 (2003), as well as U.S. Pat. No. 7,745,128, and several commercially available kits (see Dxs Diagnostic Innovations, Applied Biosystems, and Quest diagnostics).

Cancers that are either wild type or mutant for Ras/Raf and either wild type or mutant for PI3K/Pten are identified by known methods. For example, wild type or mutant Ras/Raf or PI3K/PTEN tumor cells can be identified by DNA amplification and sequencing techniques, DNA and RNA detection techniques, including, but not limited to Northern and Southern blot, respectively, and/or various biochip and array technologies. Wild type and mutant polypeptides can be detected by a variety of techniques including, but not limited to immunodiagnostic techniques such as ELISA, Western blot or immunocyto chemistry.

As used herein, “reduce” or “reducing” blast cell in bone marrow refers to a decrease in the amount of blast cells observed in bone marrow of a patient after administration of Structure I or a pharmaceutically acceptable salt or solvate thereof. As is understood in the art, blast cells can be measured by conventional means. Reductions in blast cells can be measured and assessed per individual or as a mean change for a group of subjects. Additionally, mean reductions in blast cells can be measured and assessed for a group of treated subjects as a mean change from baseline and/or as a mean change compared with the mean change in blast cells among subjects administered a different dose of the same drug, a comparator drug and/or placebo.

As used herein “eliminate” or “eliminating” blast cells in bone marrow refers to reducing the amount of blast cells in bone marrow from a subject to a level that is not easily detectable by conventional analytical means used in the art.

Thus, the present invention also provides methods of reducing or eliminating blast cells in bone marrow in a human having leukemia comprising administering to said human a pharmaceutical composition comprising at least one MEK inhibitor comprising a compound of Structure (I):

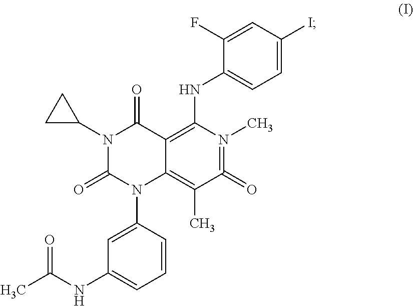

or a pharmaceutically acceptable salt or solvate thereof. In some aspects, blasts cells in bone marrow are reduced to below about 5%. In some aspects, the human has at least one Ras protein mutation in at least one leukemia cell. The Ras protein mutation may be in K-ras, H-ras or N-ras. In another aspect, methods are provided further comprising administering at least one of the following to the human having leukemia: Compound B or a pharmaceutically acceptable salt, an mTOR inhibitor, rapamycin, everolimus, deforolimus, and temsirolimus.

Also provided by the present invention are methods of treating a human having a cancer in which an abnormal number of blast cells are present in bone marrow comprising administering to said human a pharmaceutical composition comprising at least one MEK inhibitor comprising a compound of Structure (I):

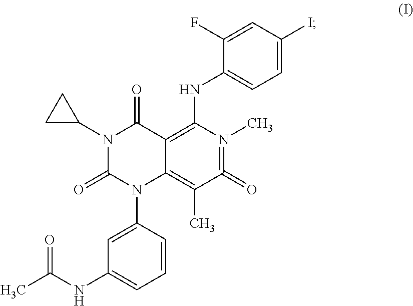

or a pharmaceutically acceptable salt or solvate thereof;
monitoring blast cells from bone marrow in said human,
genotyping for at least one Ras mutation from at least one tumor cell from said human; and
administering at least one additional dose of said pharmaceutical composition comprising Structure (I) to said human if a Ras mutation is detected. The methods may further comprise correlating the detection of at least one Ras mutation with an increased response to treatment with Structure I or a pharmaceutically acceptable salt or solvate thereof.

Methods are also provided for treating a human patient having a myeloid cancer comprising determining if a sample from said patient has at least one mutation in a Ras protein or a gene encoding at least one Ras protein and treating said patient with a therapeutically effective amount of pharmaceutical composition comprising at least one MEK inhibitor comprising a compound of Structure (I):

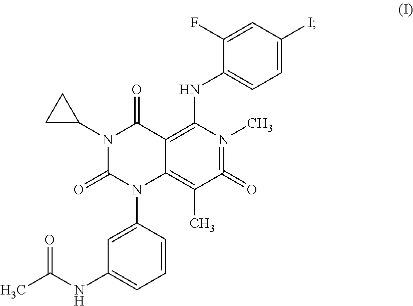

or a pharmaceutically acceptable salt or solvate thereof if it is determined that said sample has at least one mutation in a Ras protein or a gene encoding at least one Ras protein. In some instances, the patient has leukemia. The sample may be a tumor sample, blood, serum or other tissue sample. The Ras protein may have at least one mutation selected from, but not limited to, G12S, G12V, G12D, G12A, G12C, G12R, G13A, G13D, V14I, G60E, Q61H, Q61K, Q61R, T74P, E76G, E76K, E76Q and A146T.

The following examples are intended for illustration only and are not intended to limit the scope of the invention in any way.

### Example 1

### Methods

### Experimental Preparation(s)

### Cell Lines

Cancer cell lines were obtained from the American Tissue Culture Collection (ATCC) or from DSMZ (Deutsche Sammlung von Mikroorganismen and Zellkulturen), the German Resource Center for Biological Material (Braunschweig, Germany) and maintained as described by the supplier.

### Experimental Protocol(s)

### Cell Growth/Death Assay

Standard cell proliferation assays were performed on a variety of cell types according to the protocol referenced in AESOP AP5161v2. Briefly, cells were plated in 384-well plates at 500 cells/well in culture media appropriate for each cell type, supplemented with 10% FBS and 1% penicillin/streptomycin, and incubated overnight at 37° C., 5% CO₂. Cells were treated with Compound A (3-fold dilutions from 7.331 μM to 0.17 nM) and incubated at 37° C. for 72 hours. Cell growth was measured using CellTiter-Glo® reagent according to the manufacturer's protocol and signals were read on a Perkin Elmer EnVision™ reader set for luminescence mode with a 0.5-second read. Data were analyzed as described in below.

For drug combinations, 16 concentrations of 2 folds dilution of each drug were tested in a matrix for cell growth inhibition of 8 different AML cancer cell lines. Concentrations tested for Compound A and rapamycin (mTor inhibitor) were 5 μM-0.15 nM and for ara-C (DNA chain elongation inhibitor), bexarotene (retinoid X receptor activator) and sorafenib (VEGF/Raf/Kit/PDGF inhibitor) concentrations tested were 10 μM-0.3 nM. Cells were processed as described above and according to protocol AESOP AP1374V5 and data were analyzed using Excess Over Highest Single Agent (EOHSA) (Equation 1) and Excess Over Bliss formula (Equation 2) below.

### Drugs and Materials

### Compound

Compound A was synthesized by Japan Tobacco Inc. The compound was provided as a DMSO solvate powder and prepared at 5 mM in DMSO following 15 minutes sonication under 37° C. heat. Compound was kept in the dark at −20° C. and thawed out at 37° C. just before being diluted in aqueous solution at selected concentrations. Rapamycin (Sirolimus) was purchased from LC Laboratories (lot# ASW-114), 1-β-Arabinofuranosylcytosine (ara-C) was purchased from Calbiochem (cat. #251010, lot D0060258), sorafenib was synthesized and bexarotene was purchased from Chemie Tek (cat. #: 153559-49-0, lot# BEX-01A).

### Solutions, Media, and Reagents

Standard reagent solutions and cell culture media were prepared by the GSK Media Preparation Lab at the Upper Merion, Pa. site.

Gamma irradiated, heat inactivated FBS (cat. #12176-1000M) was purchased from SAFC Biosciences, Lenexa, Kans. Penicillin/streptomycin (cat. #15140) and 0.25% trypsin-EDTA (cat. #25200) were purchased from Gibco/Invitrogen, Carlsbad, Calif.

CellTiter-Glo® luminescent cell viability assay was purchased from Promega Corp., Madison, Wis.

### Data Analysis

### Concentration Response Curves for the Cell Growth/Death Assay

Results are expressed as a percentage of the t=0 value and plotted against compound concentration. The t=0 value is normalized to 100% and represents the number of cells present at the time of compound addition. The cellular response was determined for each compound using a 4- or 6-parameter curve fit of cell viability against concentration using the IDBS XLfit plug-in for Microsoft Excel software and determining the concentration required for 50% inhibition of cell growth (gIC₅₀). Background correction was made by subtraction of values from wells containing no cells.

### Analysis of Drug Combination

### Calculation of Excess Over Highest Single Agent (EOHSA)

The response (percent inhibition compared to untreated samples and normalized to media alone) of compound “A” at “a” concentration (Ra) and that of compound “B” at “b” concentration (Rb) is compared to response of the mixture of compounds “A” and “B” at concentrations “a” and “b” respectively (Rab). The equation for EOHSA is:

Rab>10% of the higher value among Ra and Rb=additive

Rab<−10% of the higher value among Ra and Rb=antagonism Equation 1

FIG. 2 summarizes the percentage of doses where “additivity” and “antagonism” was measured versus the total number of doses evaluated

### Calculation of Excess Over Bliss (EOBliss):

Using the same parameter as described above the equation for EOBliss is:

Delta=Rab−100×(Ra/100+Rb/100−Ra×Rb/10000) Equation 2

If Delta>10%, the combination is additive and if Delta<−10%, the combination is antagonistic. The EOBliss is a more stringent evaluation of drug combination than EOHSA.

### Results

### Inhibition of Cell Proliferation

Anti-proliferative activity of Compound A was tested using the CellTiter-Glo™ 3-day continuous inhibitor exposure growth-death assay. Compound A was profiled against a panel of 92 human cancer cell lines from haematological origin. Cell line sensitivities to Compound A were grouped as sensitive (gIC₅₀<200 nM), intermediate (gIC₅₀ 200 nM-2 μM), and resistant (gIC₅₀>2 μM).

Compound A potently (gIC₅₀<200 nM) inhibited proliferation of 87% (13 out of 15) and 83% (5 out of 6) of cancer cell lines from Acute Monocytic/Myelogenous Leukemia (AML) and Chronic Myelogenous Leukemia (CML) origin, respectively. However, Compound A generally showed poor to no activity against B cell leukemia, B cell lymphoma and Burkitt's lymphoma.

Combination of MEK Inhibitor with Clinically Active AML Drugs on AML Cancer Cell Lines

Growth inhibition of eight haematopoietic cancer cell lines form AML origin (F-36P, GDM-1, HEL92.1.7, ML-2, MV-4-1, OCI-AML2, OCI-AML3 and PLB985) was tested following simultaneous exposure to MEK inhibitor, Compound A, in combination with ara-C, bexarotene, rapamycin or sorafenib for 3 days. Excess Over Highest Single Agent (EOHSA) and Excess Over Bliss (EOBliss) were calculated for each drug combination and used to quantitate the efficacy of the combination. While a 16 by 16 drug matix (a total of 256 drug combinations) was tested, only physiologically relevant drug combinations were evaluated (up to 3× the IC50 for each drug); <160 nM concentrations were evaluated for Compound A and rapamycin, <310 nM concentrations for ara-C and bexarotene, and up to 10 μM concentrations were evaluated for sorafenib. In some instances, the antagonism observed at low concentration of one of the two compounds was not included in the final analysis as from the original data it was qualified as an artefact of the calculation employed or due to a plate edge effect.

The results presented in FIG. 2 demonstrate that drug combinations between Compound A and rapamycin were additive by EOHSA and EOBliss in most AML cell lines tested. For more than 60% of AML cell lines tested (5 out of 8) an EOHSA>10% was observed in more than 40% of all drug combinations tested and an EOBliss (Delta)>10% was observed in more than 30% of all combinations tested. The beneficial effect was mainly observed at concentrations from 78 nM-0.62 nM for each drug. Using this combination, only one cell line (GDM-1) demonstrated antagonism (<10% EOHSA) in >10% of drug combinations tested. As expected little additivity and antagonism were observed using this combination against HEL.92.1.7, a Compound A insensitive cell line.

For the combination of ara-C with Compound A, more than 60% of AML cell lines (5 out of 8) had an EOHSA>10% in >30% of all drug combinations tested and an EOBliss>10% in more than 20% of all combinations tested. The combination of ara-C with Compound A was slightly less additive and more antagonistic than that of Compound A and rapamycin.

The combination of bexarotene and Compound A is similar to that of Compound A and ara-C, with 5 of the 8 AML cell lines had an EOHSA>10% in more than 30% of all drug combinations tested and an EOBliss>10% in more than 20% of all combination tested.

The combination of Compound A and sorafenib caused similar level of additivity than that of ara-c or bexarotene with Compound A, however sorafenib showed the highest number of cell lines tested (50%) with EOBliss<−20%.

### Discussion

Compound A had increased activity against haematopoietic cancer cell lines from AML and CML origins compared to haematopoietic cancer cells from other origins such as acute lymphoblastic lymphoma (ALL), B cell leukaemia/lymphoma and Burkitt's lymphoma. Based on these results, sensitivity of 8 cancer cell lines from AML origin was determined following treatment with MEK inhibitor in combination with other clinically active agents. The results demonstrated that combinations of MEK inhibitor Compound A with rapamycin was generally additive at clinically relevant concentrations, with very few drug combinations causing antagonism. The drug combination of Compound A with ara-C, bexarotene and sorafenib caused similar additivity, however less than that of MEK inhibitor with rapamycin.

In summary, these results demonstrate that AML and CML are MEK inhibitor sensitive haematopoietic cancer cell lines and combination of Compound A with rapamycin, ara-C, bexarotene and sorafenib on AML cell lines offer advantages over each single agent alone.

### Example 2

Subjects were enrolled in a Phase I/II open-label, dose-escalating study. Entry criteria included subjects with relapsed/refractory leukemia for which no standard therapies are anticipated to result in durable remission. Subjects with poor-risk myelodysplasia (MDS) [i.e., refractory anemia with excess blasts (RAEB-1 or RAEB-2) by WHO classification] and chronic myelomonocytic leukemia (CMML) were also eligible. Relapsed/refractory leukemias include acute non-lymphocytic leukemia (AML) by WHO classification, acute lymphocytic leukemia (ALL), chronic lymphocytic leukemia (CLL), or chronic myelogenous leukemia (CML) in blast crisis. Subjects with agnogenic myeloid metaplasia (AMM) were also eligible.

Subjects received a pharmaceutical formulation comprising Structure I (Compound A) or a pharmaceutically acceptable salt or solvate thereof at a starting dose of 1 mg/day. Subjects received a dose for 4 weeks. Dose was escalated as tolerability was assessed with increases of about 50%.

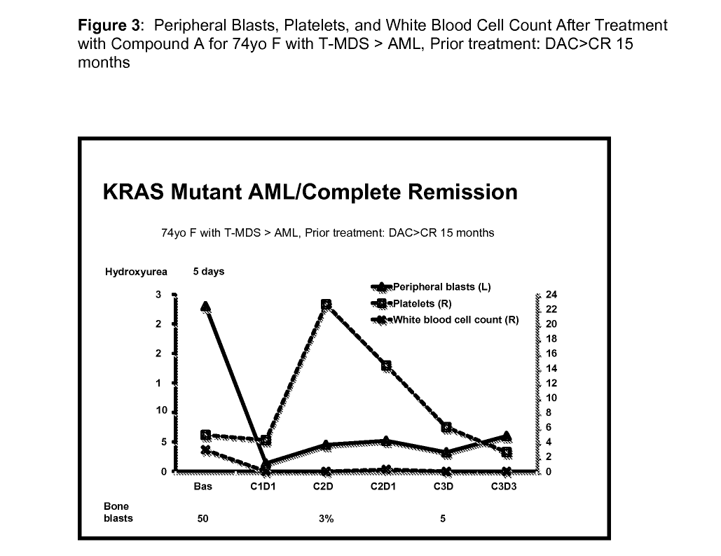

A first 74-year-old female subject with relapsed myeloid malignancy entered into the study with a two year history of chronic myelomonocytic leukemia (CMML) and acute myeloid leukemia arising from her CMML. This subject had pretreatment blast cell count from bone marrow on Days −2 and −1 of 11.0 and 9.0, respectively. Pretreatment absolute neutrophil count (ANC) was measured as 15 and 14 on Day-2 and Day-1, respectively. The subject had a blast cell count of 30.0 on Day 1 of treatment. At baseline this patient had a large blast population of 50% that were noted as large in size and showed indented and convoluted nuclear contour, fine chromatin, one or two nucleoli and finely vacuolated cytoplasm; no auer rods were identified. Blast cell count was not detectable after Day 10 (See FIG. 3). Tumor samples from this subject showed a mutation in K-ras (G12A). After receiving one month of treatment, this subject had blast cells from bone marrow of 3% with no morphologic or immunophenotypic evidence of acute leukemia. Additionally, the subject was categorized as complete remission as platelet count (PLT) increased above 100×10⁹/L by Day 29. ANC decreased after the start of treatment, but increased to above 1×10⁹/L on Day 22 and remained above 1×10⁹/L until Day 29, meeting complete remission criteria. After two months of treatment the subject had blast cells from bone marrow of about 5%. Her bone marrow diagnosis at this time was acute myeloid leukemia with 9% blast in limited aspirate smears. This patient received Compound A at a dose of 2 mg per day and had a complete remission for at least 4 weeks.

Measurements from bone marrow relating to complete remission criteria through Day 29 for said subject are presented in Table 6 below by study day.

TABLE 6

| | Treatment Day | | | | | | | | | | | | |
|---|---|---|---|---|---|---|---|---|---|---|---|---|---|
| | −2 | −1 | 1 | 2 | 3 | 7 | 10 | 13 | 15 | 17 | 22 | 24 | 29 |
| PLT | 66 | 48 | 48 | 57 | 77 | 98 | 24 | 26 | 43 | 63 | 91 | 98 | 227 |
| Blast cells | 11.0 | 9.0 | 30.0 | 17.0 | 7.0 | 6.0 | . . . | . . . | . . . | . . . | . . . | . . . | . . . |
| ANC | 15.0 | 14.6 | 12.8 | 8.3 | 5.1 | .41 | .58 | .31 | .35 | .42 | 1.4 | 2.1 | 3.2 |

A second 75-year-old female subject entered the study with persistent acute myeloid leukemia, with blasts of 33%. Blast cells from bone marrow at baseline were noted as markedly increased, intermediate to large in size with oval to slightly irregular nuclei, dispersed chromatin, distinct nucleoli and small amounts of agranular to sparsely granular cytoplasm. Many blasts were present in small cohesive clusters, enriched at the feathered edge of the smears. After two months of treatment with Compound A, the subject showed 3% blast cells in bone marrow and was diagnosed as no evidence of leukemia. This subject was found to have at least one mutation in N-ras (G12S).

A third 79-year-old male subject entered the study with persistent acute leukemia, with 14% blasts at baseline. Blasts were large with round, oval or irregular nuclei, abundant basophilic cytoplasm and fine cytoplasmic granule comprised 14% of the differential. Mature monocytes were also increased. After one month of treatment the subject showed 3% blasts in bone marrow with no morphologic evidence of acute leukemia in hypocellular bone marrow. This patient completed through 28 days of treatment with Compound A. This subject was found to have at least one mutation in N-ras.

### Example 3

An interim analyses was performed on efficacy outcomes for 67 patients treated with Compound A for one or more of the following diseases: ALL, AML, MDS, and/or CMML. Of the 67 patients, 7 patients were not evaluable for clinical activity because of unknown response or insufficient follow-up. Thirty-five patients with AML or MDS had at least one NRas or KRas mutation. Thirty patients with AML, MDS or CMML were Ras wild type or had an unknown genotype. Two patients with CMML had N-Ras or K-Ras mutations.

Clinical activity as defined by the criteria described herein is shown for these patients as part of the interim analyses in the following table, Table 7.

TABLE 7

| | AML/MDS | | AML/MDS/CMML | | CMML |
|---|---|---|---|---|---|
| | N or KRAS | | RAS wt or | | N or KRAS |
| Best response, | mutated | | unknown | | mutated |
| n (%) | (n = 35) | | (n = 30) | | (n = 2) |
| CR/CRp/ | 4 | (12) | 0 | | 1 (50) |
| Marrow CR | | | | | |
| MLFS* | 3 | (9) | 0 | | 0 |
| PR | 2 | (6) | 1 | (3) | 0 |
| HI/HI-N/HI-P | 3 | (9) | 5 | (16) | 0 |
| SD | 18 | (51) | 6 | (20) | 1 (50) |
| PD | 2 | (6) | 14 | (47) | 0 |
| Not evaluable^{†} | 3 | (9) | 4 | (13) | 0 |
| ORR | 9 | (26) | 1 | (3) | 1 (50) |

| | | |
|---|---|---|
| (95% CI 13-43) | (95% CI < 1-17) | (95% CI < 1-99) |

CR = complete remission or complete response; PR = partial remission or partial response; HI = hematologic improvement; HI-E = erythroid response; HI-P = Platelet response; HI-N = Neutrophil response; SD = stable disease; PD = progressive disease
*MLFS = morphologic leukemia-free state.
^{†}7 pts not evaluable: have unknown response or insufficient follow-up
ORR = Overall response rate = CR/CRp/Marrow CR/MLFS/PR
CRp: As per CR but platelet count <100 × 10⁹/L.

For AML/MDS Ras mutated cohort, 35 patients are enrolled and 9 responders are observed. Predictive probability of rejecting H0 at the end of the trial is >0.99 with a strong efficacy signal.

Median duration on study for Responders (CR/CRp/Marrow CR/MLFS/PR) for patients with AML and/or MDS and at least one RAS mutation is 16.3 weeks while median duration on study for SD patients is 8 weeks. Median duration on study for the partial responders with AML, MDS, and/or CMML and either RAS WT or unknown genotype is: 34.1 weeks while median duration on study for SD pts: 8.2 weeks.

Preliminary efficacy from AML/MDS-NRAS or KRAS mutated group from statistical point of view shows probability RR>15% is high and efficacy signal estimation: >25% ORR is 0.52. The response rate in ras mutant AML patients was notable with a duration of study over 10 weeks.

While the preferred embodiments of the invention are illustrated by the above, it is to be understood that the invention is not limited to the precise instructions herein disclosed and that the right to all modifications coming within the scope of the following claims is reserved.

We claim:

1. A method of treating a mammal having cancer comprising detecting at least one mutation in a Ras protein or a gene encoding at least one Ras protein from at least one tumor cell from said mammal and treating said mammal having at least one mutation in at least one Ras protein or a gene encoding at least one Ras protein with a pharmaceutical composition comprising at least one MEK inhibitor comprising a compound of Structure (I): 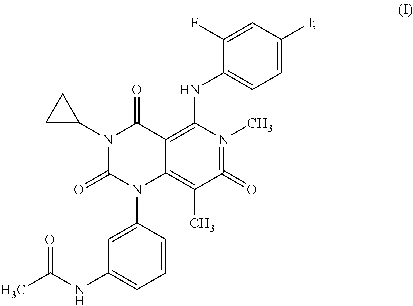
or a pharmaceutically acceptable salt or solvate thereof.

2. The method of claim 1, wherein said mammal is a human.

3. The method of claim 1 or 2, wherein said cancer is a myeloid malignancy cancer.

4. The method of any one of claims 1 to 3, wherein said cancer is leukemia.

5. The method of claim 5 wherein said cancer is selected from: acute lymphocytic leukemia, acute non-lymphocytic leukemia, acute myeloid leukemia, chronic lymphocytic leukemia, chronic myeloid leukemia, and chronic myelomonocytic leukemia.

6. The method according to any one of claims 2 to 5 wherein the human has agnogenic myeloid metaplasia.

7. The method of any one of claims 1 to 6 wherein said cancer is relapsed or refractory.

8. The method of claim 1 wherein said cancer is selected from melanoma, pancreatic cancer, colorectal cancer, and non-small cell lung carcinoma.

9. The method of any one of claims 1 to 8 wherein Structure (I) is in the sodium salt form.

10. The method of any one of claims 1 to 9 wherein Structure (I) is in the form of a dimethyl sulfoxide solvate.

11. The method of any one of claims 1 to 10 further comprising administering at least one Braf inhibitor to said human.

12. The method of claim 11 wherein said Braf inhibitor comprises a compound of Structure (II): 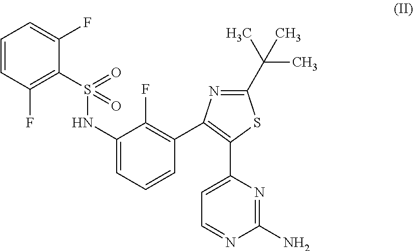 [SMILES: CC(C)(C)c1nc(-c2cccc(NS(=O)(=O)c3c(F)cccc3F)c2F)c(-c2ccnc(N)n2)s1]
or a pharmaceutically acceptable salt or solvate thereof.

13. The method of any one of claims 1 to 12 further comprising administering at least one mTOR inhibitor to said mammal.

14. The method of claim 13 wherein said mTOR inhibitor is selected from: rapamycin, everolimus, deforolimus, and temsirolimus.

15. The method of any one of claims 1 to 12 further comprising administering at least one of the following to said mammal: rapamycin, ara-C, bexarotene and sorafenib.

16. The method of any one of claims 1 to 15 wherein the mutation in at least one Ras protein or gene encoding at least one Ras protein is in K-ras, H-ras or N-ras.

17. The method of any one of claims 1 to 16 wherein the mutation in at least one gene encoding at least one Ras protein is in exon 2 or 3.

18. The method of anyone of claims 1 to 17 wherein, the gene encoding at least one Ras protein has a mutation in at least one of ras codons 12, 13, 14, 59, 60, 61, 76, or 146.

19. The method of any one of claims 1 to 18 wherein at least Ras protein has at least one mutation selected from G12S, G12V, G12D, G12A, G12C, G12R, G13A, G13R, G13D, V14I, G60E, Q61H, Q61K, Q61R, T74P, E76G, E76K, E76Q and A146T.

20. The method of any one of claims 1 to 18 wherein said at least one Ras protein is K-ras with a mutation at G12A.

21. The method of any one of claims 1 to 19 wherein said at least one Ras protein is N-ras with a mutation at G12S.

22. The method of any one of claims 1 to 21 wherein said tumor cell also has at least one Braf mutation.

23. The method of claim 22 wherein said Braf mutation is selected from: R462I, I463S, G464V, G464E, G466A, G466E, G466V, G469A, G469E, D594V, F595L, G596R, L597V, L597R, T599I, V600E, V600D, V600K, V600R, T119S, and K601E.

24. The method of any one of claims 3 to 7 or 9 to 23 wherein said human shows complete remission of myeloid malignancy after administration of said pharmaceutical composition comprising Structure I or a pharmaceutically acceptable salt or solvate thereof.

25. The method of claim 24 wherein complete remission is measured by said human being free of all symptoms of leukemia and having an absolute neutrophil count 1×10⁹/L and platelet count 100×10⁹/L and normal marrow differential with 5% blast cells.

26. The method of claim 24 or 25 wherein said human has no detectable blast cells in bone marrow after receiving at least one week of treatment with a pharmaceutical composition comprising Structure (I) of a pharmaceutically acceptable salt or solvate thereof.

27. A method according to any one of claims 1 to 26 where the amount of the Structure I of a pharmaceutically acceptable salt or solvate thereof is an amount selected from 0.125 mg to 10 mg.

28. The method according to any one of claims 1 to 26 wherein the amount of Structure I of a pharmaceutically acceptable salt or solvate thereof is administered once daily at a dose selected from: 1.0 mg/day, 1.5 mg/day and 2.0 mg/day.

29. A method according to any one of claims 11 to 28 wherein the amount BRAF inhibitor is an amount selected from 75 mg to 1,000 mg.

30. A method according to any one of claims 11 to 29 wherein the pharmaceutical composition comprising Structure I or a pharmaceutically acceptable salt or solvate thereof and the pharmaceutical composition comprising at least one Braf inhibitor are administered separately.

31. A method according to any one of claims 11 to 29 wherein the pharmaceutical composition comprising Structure I or a pharmaceutically acceptable salt or solvate thereof is administered at the same time as the pharmaceutical composition comprising Structure II or a pharmaceutically acceptable salt or solvate thereof.

32. A method of reducing or eliminating blast cells in bone marrow in a human having leukemia comprising administering to said human a pharmaceutical composition comprising at least one MEK inhibitor comprising a compound of Structure (I): 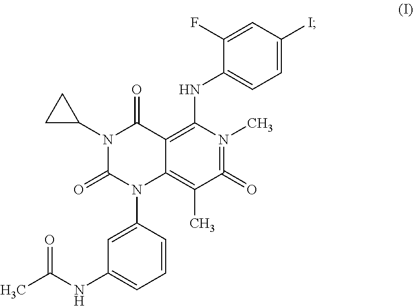
or a pharmaceutically acceptable salt or solvate thereof.

33. The method of claim 32 wherein said blasts cells in bone marrow is reduced to below about 5%.

34. The method of claim 32 or 33 wherein a leukemia cell isolated from said human has at least one Ras protein mutation.

35. The method of any one of claims 32 to 34 wherein said Ras protein mutation is in K-ras, H-ras or N-ras.

36. The method of any one of claims 32 to 35 further comprising administering at least one of the following to said human: Compound B or a pharmaceutically acceptable salt, rapamycin, everolimus, deforolimus, and temsirolimus.

37. A method of treating a human having a cancer in which an abnormal number of blast cells are present in bone marrow comprising administering to said human a pharmaceutical composition comprising at least one MEK inhibitor comprising a compound of Structure (I): 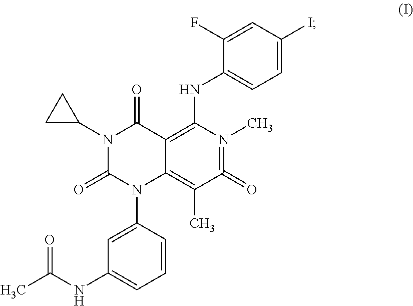
or a pharmaceutically acceptable salt or solvate thereof;
monitoring blast cells from bone marrow in said human,
genotyping for at least one Ras mutation from at least one tumor cell from said human; and
administering at least one additional dose of said pharmaceutical composition comprising Structure (I) to said human if a Ras mutation is detected.

38. The method of claim 37 further comprising correlating the detection of at least one Ras mutation with an increased response to treatment with Structure I or a pharmaceutically acceptable salt or solvate thereof.

39. A method of treating a human patient having a myeloid cancer comprising determining if a sample from said patient has at least one mutation in a Ras protein or a gene encoding at least one Ras protein and treating said patient with a therapeutically effective amount of pharmaceutical composition comprising at least one MEK inhibitor comprising a compound of Structure (I): 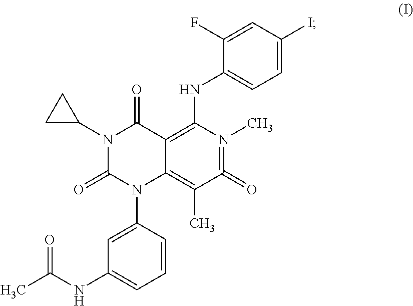
or a pharmaceutically acceptable salt or solvate thereof if it is determined that said sample has at least one mutation in a Ras protein or a gene encoding at least one Ras protein.

40. The method of claim 39 wherein the patient has leukemia.

41. The method of claim 39 wherein said sample is a tumor sample.

42. The method of claim 39 wherein said sample is a blood sample.

43. The method any one of claims 39 to 42 wherein at least Ras protein has at least one mutation selected from G12S, G12V, G12D, G12A, G12C, G12R, G13A, G13D, G13R, V14I, G60E, Q61H, Q61K, Q61R, T74P, E76G, E76K, E76Q and A146T.
